(19) United States
(12) Patent Application Publication
(10) Pub. No.: US 2026/0347864 A1
(43) Pub. Date: Nov. 5, 2026

(54) PLANT REGULATORY ELEMENTS AND USES THEREOF

(71) Applicant: Monsanto Technology LLC, St. Louis, MO (US)

(72) Inventors: Ian W. Davis, Grover, MO (US); Matthew S. Marengo, Wildwood, MO (US); Ervin D. Nagy, Lake St. Louis, MO (US)

(21) Appl. No.: 19/591,136

(22) PCT Filed: Dec. 2, 2024
(86) PCT No.: PCT/US2024/058071

§ 371 Date: May 27, 2026

Related U.S. Application Data
(60) Provisional application No. 63/605,883, filed on Dec. 4, 2023.

(51) Int. Cl. C12N 15/82; C12N 9/22; C12N 15/11

(52) U.S. Cl. CPC C12N 15/8216; C12N 9/226; C12N 15/111; C07K 2319/02; C12N 2310/20

(57) ABSTRACT

The disclosure provides novel synthetic small nuclear RNA (snRNA) promoters which are useful for CRISPR-mediated targeted gene modifications in plants. The disclosure also provides methods and compositions for use for the snRNA promoters driving expression of guide RNAs and non-coding RNAs for development of plants and plant cells comprising modified genomes.

### REFERENCE TO RELATED APPLICATIONS

This application claims the benefit of U.S. provisional application No. 63/605,883, filed Dec. 4, 2023, herein incorporated by reference in its entirety.

### INCORPORATION OF SEQUENCE LISTING

The sequence listing that is contained in the file named “MONS583WO_ST26.xml”, which is 43,754 bytes (as measured in Microsoft Windows®) and was created on Nov. 26, 2024, is filed herewith by electronic submission and is incorporated by reference herein.

### FIELD

The disclosure relates to the field of biotechnology. More specifically, the disclosure provides novel synthetic plant promoters beneficial for the expression of, for instance, non-protein-coding small RNAs for CRISPR-mediated genome modification.

### BACKGROUND

Site-specific recombination has potential for application across a wide range of biotechnology-related fields. Meganucleases, zinc finger nucleases (ZFNs), and transcription activator-like effector nucleases (TALENs) containing a DNA-binding domain and a DNA cleavage domain enable genome modification. While meganucleases, ZFNs, and TALENs, are effective and specific, these technologies require generation through protein engineering of one or more components for each genomic site chosen for modification. Advances in application of clustered, regularly interspaced, short palindromic repeats have illustrated a method of targeted genome modification that has the advantage of being quick to engineer.

The Clustered Regularly Interspaced Short Palindromic Repeats (CRISPR) system constitutes an adaptive immune system in prokaryotes that targets endonucleolytic cleavage of invading phage. The system is composed of a protein component (Cas) and a guide RNA (gRNA) that targets the Cas protein to a specific locus for endonucleolytic cleavage. This system has been successfully engineered to target specific loci for endonucleolytic cleavage of mammalian, zebrafish, drosophila, nematode, bacteria, yeast, and plant genomes.

It is preferable that the DNA sequence encoding the guide RNA that targets the Cas protein to a specific locus for endonucleolytic cleavage be transcribed by RNA Polymerase III which transcribes small nuclear RNAs (snRNAs). Native promoters, such as the U6 snRNA promoters, are often used to drive expression of gRNAs. Multiplex targeting experiments often rely on the same promoter driving each of the gRNAs. This sequence redundancy amongst cassettes can lead to technical problems when cloning or maintaining plasmids that comprise multiple U6/gRNA cassettes, such as recombination events or deletions. Having multiple snRNA promoters with diverse DNA sequences will help alleviate this technical issue. Thus, the inventors disclose herein novel synthetic snRNA promoters that have little sequence homology with known native U6 snRNA promoters and each other. These novel synthetic snRNA promoters are capable of driving the expression of RNA polymerase III transcripts, such as gRNAs, in plant cells.

### SUMMARY

This section provides a general summary of the disclosure and is not a comprehensive disclosure of its full scope or all of its features.

Provided herein are gene regulatory elements for use in plants. Several embodiments relate to recombinant DNA molecules comprising the regulatory elements. Also provided are transgenic plant cells, and plants, comprising the regulatory elements. In one embodiment, the regulatory elements are operably linked to a transcribable DNA molecule. In certain embodiments, the transcribable DNA molecule may be heterologous with respect to the regulatory element. Thus, a regulatory element DNA sequence provided herein may, in particular embodiments, be defined as operably linked to a heterologous transcribable DNA molecule. Several embodiments relate to methods of using the regulatory elements and making and using the recombinant DNA molecules comprising the regulatory elements, and the transgenic plant cells, and plants comprising the regulatory elements operably linked to a transcribable DNA molecule.

In one aspect, provided herein is a recombinant DNA molecule comprising a DNA sequence selected from the group consisting of: (a) a sequence with at least 85% sequence identity to any of SEQ ID NOs: 1-16; (b) a sequence comprising any of SEQ ID NOs: 1-16; and (c) a fragment of any of SEQ ID NOs: 1-16, wherein the fragment comprises gene regulatory activity. In one embodiment, the recombinant DNA molecule has at least 90 percent sequence identity to the DNA sequence of any of SEQ ID NOs: 1-16. In another embodiment, the recombinant DNA molecule has at least 95 percent sequence identity to the DNA sequence of any of SEQ ID NOs: 1-16. In yet another embodiment, the DNA sequence of the recombinant DNA molecule comprises gene regulatory activity.

In one embodiment, the DNA sequence comprised in the recombinant DNA molecule comprises a synthetic small nuclear RNA (snRNA) promoter. In another embodiment, the DNA sequence comprised in the recombinant DNA molecule may be operably linked to a heterologous transcribable DNA molecule. In yet another embodiment, the heterologous transcribable DNA molecule encodes a non-coding RNA. In a further embodiment, said non-coding RNA may be selected from the group consisting of: a guide RNA (gRNA), a single-guide RNA (sgRNA), a crRNA, a pre-crRNA, a tracrRNA, a PEgRNA, a target-allele guide RNA (tagRNA), a microRNA (miRNA), a miRNA precursor, a small interfering RNA (siRNA), a small RNA (22-26 nt in length) and precursor encoding same, a heterochromatic siRNA (hc-siRNA), a Piwi-interacting RNA (piRNA), a hairpin double-strand RNA (hairpin dsRNA), a trans-acting siRNA (ta-siRNA), and a naturally occurring antisense siRNA (nat-siRNA). In another embodiment, said non-coding RNA may be a gRNA.

In another aspect, provided herein is a recombinant DNA construct comprising at least a first expression cassette comprising a first recombinant DNA molecule, operably linked to a DNA sequence encoding a gRNA. In one embodiment, the recombinant DNA molecule comprises a DNA sequence selected from the group consisting of: (a) a sequence with at least 85% sequence identity to any of SEQ ID NOs: 1-16; (b) a sequence comprising any of SEQ ID NOs: 1-16; and (c) a fragment of any of SEQ ID NOs: 1-16, wherein the fragment comprises gene regulatory activity. In another embodiment, the recombinant DNA construct further comprises at least a second or more expression cassettes, wherein each of the expression cassettes comprise a recombinant DNA molecule operably linked to a DNA sequence encoding a gRNA. In another embodiment, the DNA sequences encoding the gRNAs may be identical or distinct from each other. In yet another embodiment, the DNA sequences encoding the gRNAs can target the same or different target sites in a chromosome of a plant cell. The recombinant DNA molecules comprised within the expression cassettes comprised within the recombinant DNA construct may be identical or distinct from each other. In one embodiment, the recombinant DNA molecules comprised within the expression cassettes comprised within the DNA construct comprise promoter activity. In another embodiment, each expression cassette comprised within the recombinant DNA construct further comprises a transcription termination sequence. In yet another aspect, the recombinant DNA construct further comprises a DNA sequence encoding a promoter operably linked to a DNA sequence encoding a Type I CRISPR-associated protein, a Type II CRISPR-associated protein, a Type III CRISPR-associated protein, a Type IV CRISPR-associated protein, Type V CRISPR-associated protein, or a Type VI CRISPR-associated protein. In one embodiment, the DNA sequence encoding the CRISPR-associated protein may be further operably linked to at least one DNA sequence encoding a nuclear localization sequence (NLS). Further, the CRISPR-associated protein may be selected from the group consisting of: Cas1, Cas1B, Cas2, Cas3, Cas4, Cas5, Cas6, Cas7, Cas8, Cas9, Cas10, Cas12a, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, and Mad7. In one embodiment, the CRISPR-associated protein may be Cas12a.

In another aspect provided herein is a plant cell comprising the recombinant DNA molecule or the recombinant DNA construct. The cell plant cell may be a monocotyledonous plant cell or dicotyledonous plant cell.

### DESCRIPTION OF THE SEQUENCES

SEQ ID NO:1 is a DNA sequence of the synthetic snRNA promoter, P-GSP2241.

SEQ ID NO:2 is a DNA sequence of the synthetic snRNA promoter, P-GSP2242.

SEQ ID NO:3 is a DNA sequence of the synthetic snRNA promoter, P-GSP2243.

SEQ ID NO:4 is a DNA sequence of the synthetic snRNA promoter, P-GSP2247.

SEQ ID NO:5 is a DNA sequence of the synthetic snRNA promoter, P-GSP2248.

SEQ ID NO:6 is a DNA sequence of the synthetic snRNA promoter, P-GSP2249.

SEQ ID NO:7 is a DNA sequence of the synthetic snRNA promoter, P-GSP2250.

SEQ ID NO:8 is a DNA sequence of the synthetic snRNA promoter, P-GSP2251.

SEQ ID NO:9 is a DNA sequence of the synthetic snRNA promoter, P-GSP2253.

SEQ ID NO: 10 is a DNA sequence of the synthetic snRNA promoter, P-GSP2254.

SEQ ID NO:11 is a DNA sequence of the synthetic snRNA promoter, P-GSP2255.

SEQ ID NO: 12 is a DNA sequence of the synthetic snRNA promoter, P-GSP2256.

SEQ ID NO:13 is a DNA sequence of the synthetic snRNA promoter, P-GSP2258.

SEQ ID NO: 14 is a DNA sequence of the synthetic snRNA promoter, P-GSP2259.

SEQ ID NO: 15 is a DNA sequence of the synthetic snRNA promoter, P-GSP2260.

SEQ ID NO: 16 is a DNA sequence of the synthetic snRNA promoter, P-GSP2261.

SEQ ID NO: 17 is a DNA sequence of a guide RNA, gRNA_Zm7.1c.

SEQ ID NO: 18 is a DNA sequence of a guide RNA, gRNA_Bmr3_3170.

SEQ ID NO:19 is a DNA sequence of an expression element group (EXP), EXP-Zm.UbqM1:1:9 comprised of a promoter, leader, and intron derived from a Zea mays ssp. mexicana ubiquitin gene.

SEQ ID NO:20 is a DNA sequence encoding a nuclear targeted Cas12a protein, Cas12a NLS.

SEQ ID NO:21 is a DNA sequence of the 3′ UTR, T-Os.LTP: 2.

SEQ ID NO:22 is an amino acid sequence of a nuclear targeted Cas12a protein encoded by SEQ ID NO:20.

SEQ ID NO:23 is a DNA sequence of the Zea mays Zm7 genomic region targeted for genome editing.

SEQ ID NO:24 is a DNA sequence of the Zea mays brown midrib 3 (Bmr3) genomic region targeted for genome editing.

### DESCRIPTION OF THE FIGURES

FIG. 1 is a graphical representation of the mean percentage insertion/deletion (Indel) introduced into the Zm7.1c target site within the Zea mays Zm7 genomic region of corn leaf protoplasts, corresponding to each of the synthetic snRNA promoters.

FIG. 2 is a graphical representation of the mean percentage insertion/deletion (Indel) introduced into the Bmr3_3170 target site within the Zea mays brown midrib 3 (Bmr3) genomic region of corn leaf protoplasts, corresponding to each of the synthetic snRNA promoters.

### DETAILED DESCRIPTION

Recombinant DNA molecules, fragments and variants thereof, and their corresponding DNA sequences are provided. As used herein, the terms “DNA”, “DNA polynucleotide”, “DNA molecule” and “nucleic acid molecule” refer to a deoxyribonucleic acid (DNA) molecule. A DNA polynucleotide may be described by convention from the 5′ (upstream) end to the 3′ (downstream) end. A DNA polynucleotide may be of genomic or synthetic origin and/or comprise a recombinant or heterologous DNA polynucleotide or sequence. As used herein, the term “DNA sequence” refers to the nucleotide sequence of a DNA polynucleotide, i.e. the sequence of consecutive nucleotides in the DNA molecule. Examples of DNA sequences disclosed herein include the DNA sequences of SEQ ID NOs: 1-24, or variants or fragments thereof, or the reverse complements of any of SEQ ID NOs: 1-24. As used herein in reference to nucleotides of a DNA polynucleotide or DNA sequence or molecule, the terms “consecutive” and “contiguous” are interchangeable and synonymous and refer to linked nucleotides in a DNA polynucleotide or DNA sequence, strand or molecule without any gap or interruption between them.

Provided herein are recombinant DNA molecules that comprise novel synthetic snRNA (small nuclear RNA) promoters having gene regulatory activity in plants. The nucleotide sequences of these snRNA promoters are provided as SEQ ID NOs: 1-16. These snRNA promoters are capable of affecting the expression of non-coding RNAs, such as guide RNAs, in plant tissues, and therefore regulating expression of an operably linked sequence encoding the non-coding RNA in plants. Also provided are methods of modifying, producing, and using recombinant DNA molecules which contain the provided snRNA promoters. Also provided are compositions that include transgenic plant cells, plants, plant parts, and seeds containing the snRNA promoters of the disclosure, and methods for preparing and using the same.

In some embodiments, variants of a snRNA promoter selected from any of SEQ ID NOs: 1-16 are provided. As used herein, the term “variant” refers to a second DNA molecule, such as a regulatory element, that is in composition similar, but not identical to, a first DNA molecule, and wherein the second DNA molecule still maintains the general functionality, e.g., the same or similar expression pattern, for instance through more or less equivalent transcriptional activity, of the first DNA molecule. A variant may be a shorter or truncated version of the first DNA molecule or an altered version of the sequence of the first DNA molecule, such as one with different restriction enzyme sites and/or internal deletions, substitutions, or insertions. A “variant” can also encompass a regulatory element having a nucleotide sequence comprising one or more modifications for example, a substitution, duplication, deletion, and/or insertion of one or more nucleotides of a reference sequence, wherein the derivative regulatory element has more or less or equivalent transcriptional or translational activity than the corresponding parent regulatory DNA molecule or element. Regulatory element “variants” will also encompass variants arising from mutations that naturally occur in bacterial and plant cell transformation. In some embodiments, a DNA molecule sequence provided as any of SEQ ID NOs: 1-16 may be used to create variants that are similar in composition, but not identical to, the DNA sequence of the original regulatory element i.e., any of SEQ ID NOs: 1-16, while still maintaining the general functionality, e.g., the same or similar expression pattern, of the original regulatory element. In some embodiments, a variant comprising a sequence that, when optimally aligned to a reference sequence, provided herein as any of SEQ ID NOs: 1-16, has at least about 85 percent identity, at least about 86 percent identity, at least about 87 percent identity, at least about 88 percent identity, at least about 89 percent identity, at least about 90 percent identity, at least about 91 percent identity, at least about 92 percent identity, at least about 93 percent identity, at least about 94 percent identity, at least about 95 percent identity, at least about 96 percent identity, at least about 97 percent identity, at least about 98 percent identity, or at least about 99 percent identity to the reference sequence and having promoter activity as disclosed herein are provided. Variants of any of SEQ ID NOs: 1-16 may have the activity of the base sequence, for example the promoter activity of the base sequence. Production of such variants is well within the ordinary skill of the art in light of the disclosure and is contemplated herein.

In some embodiments, fragments of a snRNA promoter selected from any of SEQ ID NOs: 1-16 are provided comprising at least about 50, at least about 75, at least about 95, at least about 100, at least about 125, at least about 150, at least about 175, at least about 200, at least about 225, at least about 250, at least about 275, or longer, of a DNA molecule having gene regulatory and/or promoter activity as disclosed herein. In certain embodiments, provided are fragments of a snRNA promoter provided herein, having gene regulatory activity. Methods for producing such fragments from a starting promoter molecule are well known in the art. Fragments of any of SEQ ID NOs: 1-16 may have the activity of the base sequence, for example the promoter activity of the base sequence.

Compositions derived from any of the promoter elements comprised within any of SEQ ID NOs: 1-16, such as internal or 5′ deletions, for example, can be produced using methods known in the art to improve or alter expression, including by removing elements that have either positive or negative effects on expression; duplicating elements that have positive or negative effects on expression; and/or duplicating or removing elements that have tissue- or cell-specific effects on expression. Compositions derived from any of the promoter elements comprised within any of SEQ ID NOs: 1-16 comprised of 3′ deletions in which the TATA box element or equivalent sequence thereof and downstream sequence is removed can be used, for example, to make enhancer elements. These enhancer elements can be operably linked to other synthetic or native snRNA promoters to enhance expression. Further deletions can be made to remove any elements that have positive or negative effects on expression. Any of the promoter elements comprised within any of SEQ ID NOs: 1-16 and fragments or enhancers derived therefrom can be used to make chimeric transcriptional regulatory element compositions.

In some embodiments, the disclosure provides novel synthetic snRNA (small nuclear RNA) promoters, and methods for their use that include expression of guide RNAs for targeted gene modification of a plant genome by Clustered Regularly Interspaced Short Palindromic Repeats (CRISPR) editing systems. For instance, the disclosure provides, in one embodiment, recombinant DNA constructs encoding at least one expression cassette including a synthetic snRNA promoter (or functional fragment or variant thereof) disclosed herein and a DNA sequence encoding one or more guide RNAs (gRNAs). Methods for causing a CRISPR system to modify a target genome are also provided, as are the genomic complements of a plant modified by the use of such a system. The disclosure thus provides tools and methods that allow one to, for example, insert, remove, or modify genes, loci, linkage blocks, and chromosomes within a plant genome.

The disclosure provides, in another embodiment, recombinant DNA constructs encoding at least one expression cassette comprising a promoter disclosed herein and a DNA sequence encoding a non-protein-coding small RNA (npcRNA). These constructs are useful for expression of npcRNA molecules.

The CRISPR system constitutes an adaptive immune system in prokaryotes that targets endonucleolytic cleavage of the DNA and RNA of invading phage (reviewed in Westra et al., Annu Rev Genet 46:311-39, 2012). There are six known types of CRISPR systems which rely on small RNAs for sequence-specific detection and targeting of foreign nucleic acids for destruction: Type I, Type II, Type III, Type IV, Type V, and Type VI. The components of the bacterial CRISPR systems are CRISPR-associated (Cas) proteins and CRISPR array(s) comprising genome-targeting sequences (protospacers) interspersed with short palindromic repeats. For CRISPR Type II systems, transcription of the spacer/repeat elements into precursor CRISPR RNA (pre-crRNA) molecules is followed by enzymatic cleavage triggered by hybridization between a trans-acting CRISPR RNA (tracrRNA) molecule and a pre-crRNA palindromic repeat. The resulting crRNA: tracrRNA molecules comprise one copy of the spacer and one scaffold that can complex with a Cas nuclease. This CRISPR/Cas complex is then directed to DNA sequences (the target strand of protospacer) complementary to the crRNA spacer sequence, where this RNA-Cas protein complex silences the target DNA through enzymatic cleavage of both strands (double-strand break; DSB).

The native bacterial type II CRISPR system requires four molecular components for targeted cleavage of exogenous DNAs: a Cas endonuclease (e.g., Cas9), a house-keeping RNaseIII, CRISPR RNA (crRNA) and trans-acting CRISPR RNA (tracrRNA). The latter two components form a dsRNA complex and bind to Cas9 resulting in an RNA-guided DNA endonuclease complex. For targeted genome modifications in eukaryotes, this system was simplified to two components: the Cas9 endonuclease and a guide-RNA (gRNA). Experiments initially conducted in eukaryotic systems determined that the RNaseIII component was not necessary to achieve targeted DNA cleavage. The minimal two component system of Cas9 with the gRNA, as the only target-specific component, enables this CRISPR system of targeted genome modification to be more cost effective and flexible than other targeting platforms such as meganucleases, Zinc finger nucleases, or TALE-nucleases which require protein engineering for modification at each targeted DNA site. Additionally, the ease of design and production of gRNAs provides the CRISPR system with several advantages for application of targeted genome modification. For example, the CRISPR/Cas system components (Cas endonuclease, gRNA, and, optionally, exogenous DNA for integration into the genome) designed for one or more genomic target sites can be multiplexed in one transformation, or the introduction of the CRISPR/Cas system components can be spatially and/or temporally separated.

As used herein, a “guide nucleic acid” or “guide RNA” or “gRNA” means a nucleic acid that comprises a spacer sequence, which is complementary to (and hybridizes to) a target DNA sequence, and a scaffold sequence, which binds to a Cas protein. In some embodiments, the scaffold comprises only the CRISPR repeat of the crRNA or, the combination of the crRNA and the tracrRNA. In some embodiments of this latter scenario, the crRNA and tracrRNA are covalently bond in a chimeric fashion, which will be referred to herein as “single-guide RNA” (or “sgRNA”). In some other embodiments, the crRNA and tracrRNA species are expressed separately, referred to herein as “dual-guide RNA” (or “dgRNA”). The spacer sequence may be linked to the 5′ end or the 3′ end of the scaffold sequence. In some embodiments, the design of the gRNA may be based on a Type I, Type II, Type III, Type IV, Type V, or Type VI CRISPR-Cas system.

In some embodiments, an array of guide RNAs are expressed from a synthetic snRNA promoter (or functional fragment or variant thereof) as described herein. In some embodiments, a synthetic snRNA promoter (or functional fragment or variant thereof) as described herein may be operably linked to more than one scaffold-spacer (and/or spacer-scaffold) sequence (e.g., 2, 3, 4, 5, 6, 7, 8, 9, 10, or more scaffold-spacer (and/or spacer-scaffold) sequences) (e.g., scaffold-spacer-scaffold, e.g., spacer-scaffold-spacer, e.g., scaffold-spacer-scaffold-spacer-scaffold-spacer-scaffold-spacer-scaffold-spacer, e.g., spacer-scaffold-spacer-scaffold-spacer-scaffold-spacer-scaffold-spacer-scaffold, and the like). In some embodiments, a guide RNA array comprises one or more tRNAs as described in WO 2016/061481. In some embodiments, the guide RNA array comprises one or more tRNAs separating the guide RNA units, wherein one gRNA unit includes one scaffold and one spacer directly bond to each other (e.g., scaffold-spacer-tRNA-scaffold-spacer, e.g., spacer-scaffold-tRNA-spacer-scaffold, e.g., scaffold-spacer-tRNA-scaffold-spacer-RNA-scaffold-spacer-tRNA-scaffold-spacer-tRNA-scaffold-spacer, e.g., spacer-scaffold-tRNA-spacer-scaffold-tRNA-spacer-scaffold-tRNA-spacer-scaffold-tRNA-spacer-scaffold, and the like). In some embodiments, a scaffold sequence is selected from the group consisting of: a repeat sequence of a Cas12a CRISPR-Cas system or a fragment thereof; a repeat sequence of a Cas12b CRISPR-Cas system or a fragment thereof; a repeat sequence of a Cas12c CRISPR-Cas system or a fragment thereof; a repeat sequence of a Cas12d CRISPR-Cas system or a fragment thereof; a repeat sequence of a Cas12e CRISPR-Cas system or a fragment thereof; a repeat sequence of a Cas9 CRISPR-Cas system or fragment thereof; a repeat sequence of a C2c1 CRISPR Cas system or a fragment thereof; a repeat sequence of a C2c3 CRISPR-Cas system or a fragment thereof; a repeat sequence of a Cas13a CRISPR-Cas system or a fragment thereof; a repeat sequence of a Cas13b CRISPR-Cas system or a fragment thereof; a repeat sequence of a Cas13c CRISPR-Cas system or a fragment thereof; a repeat sequence of a Cas13d CRISPR-Cas system or a fragment thereof; a repeat sequence of a Cas1 CRISPR-Cas system or a fragment thereof; a repeat sequence of a Cas1B CRISPR-Cas system or a fragment thereof; a repeat sequence of a Cas2 CRISPR-Cas system or a fragment thereof, a repeat sequence of a Cas3 CRISPR-Cas system or a fragment thereof; a repeat sequence of a Cas3′ CRISPR-Cas system or a fragment thereof; a repeat sequence of a Cas3″ CRISPR-Cas system or a fragment thereof; a repeat sequence of a Cas4 CRISPR-Cas system or a fragment thereof; a repeat sequence of a Cas5 CRISPR-Cas system or a fragment thereof; a repeat sequence of a Cas6 CRISPR-Cas system or a fragment thereof; a repeat sequence of a Cas7 CRISPR-Cas system or a fragment thereof; a repeat sequence of a Cas8 CRISPR-Cas system or a fragment thereof; a repeat sequence of a Cas10 CRISPR-Cas system or a fragment thereof; a repeat sequence of a Csy1 CRISPR-Cas system or a fragment thereof; a repeat sequence of a Csy2 CRISPR-Cas system or a fragment thereof, a repeat sequence of a Csy3 CRISPR-Cas system or a fragment thereof; a repeat sequence of a Cse1 CRISPR-Cas system or a fragment thereof; a repeat sequence of a Cse2 CRISPR-Cas system or a fragment thereof; a repeat sequence of a Csc1 CRISPR-Cas system or a fragment thereof; a repeat sequence of a Csc2 CRISPR-Cas system or a fragment thereof; a repeat sequence of a Csa5 CRISPR-Cas system or a fragment thereof; a repeat sequence of a Csn2 CRISPR-Cas system or a fragment thereof; a repeat sequence of a Csm2 CRISPR-Cas system or a fragment thereof; a repeat sequence of a Csm3 CRISPR-Cas system or a fragment thereof; a repeat sequence of a Csm5 CRISPR-Cas system or a fragment thereof; a repeat sequence of a Csm6 CRISPR-Cas system or a fragment thereof; a repeat sequence of a Cmr1 CRISPR-Cas system or a fragment thereof; a repeat sequence of a Cmr3 CRISPR-Cas system or a fragment thereof; a repeat sequence of a Cmr4 CRISPR-Cas system or a fragment thereof; a repeat sequence of a Cmr5 CRISPR-Cas system or a fragment thereof; a repeat sequence of a Cmr6 CRISPR-Cas system or a fragment thereof; a repeat sequence of a Csb1 CRISPR-Cas system or a fragment thereof; a repeat sequence of a Csb2 CRISPR-Cas system or a fragment thereof; a repeat sequence of a Csb3 CRISPR-Cas system or a fragment thereof; a repeat sequence of a Csx 10 CRISPR-Cas system or a fragment thereof; a repeat sequence of a Csx14 CRISPR-Cas system or a fragment thereof; a repeat sequence of a Csx15 CRISPR-Cas system or a fragment thereof; a repeat sequence of a Csx 16 CRISPR-Cas system or a fragment thereof; a repeat sequence of a Csx17 CRISPR-Cas system or a fragment thereof; a repeat sequence of a CsaX CRISPR-Cas system or a fragment thereof; a repeat sequence of a Csx1 CRISPR-Cas system or a fragment thereof; a repeat sequence of a Csx3 CRISPR-Cas system or a fragment thereof; a repeat sequence of a Csf1 CRISPR-Cas system or a fragment thereof; a repeat sequence of a Csf2 CRISPR-Cas system or a fragment thereof; a repeat sequence of a Csf3 CRISPR-Cas system or a fragment thereof; a repeat sequence of a Csf4 CRISPR-Cas system or a fragment thereof; and a repeat sequence of a Csf5 CRISPR-Cas system or a fragment thereof.

In some embodiments, a guide RNA expressed from a synthetic snRNA promoter (or functional fragment or variant thereof) as described herein may comprise more than one crRNA sequences (e.g., 2, 3, 4, 5, 6, 7, 8, 9, 10, or more crRNA sequences). In some embodiments, the guide RNA comprises one or more tRNAs separating the crRNA sequences (e.g., crRNA-tRNA-crRNA, e.g., crRNA-tRNA-crRNA-tRNA-crRNA-tRNA-crRNA-tRNA-crRNA, and the like).

In some embodiments, a guide RNA array expressed from a synthetic snRNA promoter (or functional fragment or variant thereof) as described herein may comprise more than one tracrRNA sequences (e.g., 2, 3, 4, 5, 6, 7, 8, 9, 10, or more tracrRNA sequences). In some embodiments, the guide RNA array comprises one or more tRNAs separating the tracrRNA sequences (e.g., tracrRNA-tRNA-tracrRNA, e.g., tracrRNA-tRNA-tracrRNA-tRNA-tracrRNA-tRNA-tracrRNA-tRNA-tracrRNA, and the like).

In some embodiments, a guide RNA array expressed from a synthetic snRNA promoter a (or functional fragment or variant thereof) s described herein may comprise more than one crRNA-tracrRNA (and/or tracrRNA-crRNA) sequence (e.g., 2, 3, 4, 5, 6, 7, 8, 9, 10, or more crRNA-tracrRNA (and/or tracrRNA-crRNA) sequences) (e.g., crRNA-tracrRNA-crRNA, e.g., tracrRNA-crRNA-tracrRNA, e.g., crRNA-tracrRNA-crRNA-tracrRNA-crRNA-tracrRNA-crRNA-tracrRNA-crRNA-tracrRNA, e.g., tracrRNA-crRNA-tracrRNA-crRNA-tracrRNA-crRNA-tracrRNA-crRNA-tracrRNA, and the like). In some embodiments, the guide RNA array comprises one or more tRNAs separating the crRNA and tracrRNA sequences (e.g., crRNA-tracrRNA-tRNA-crRNA-tracrRNA, e.g., tracrRNA-crRNA-tRNA-tracrRNA-crRNA, e.g., crRNA-tracrRNA-tRNA-crRNA-tracrRNA-tRNA-crRNA-tracrRNA-tRNA-crRNA-tracrRNA-tRNA-crRNA-tracrRNA, e.g., tracrRNA-crRNA-tRNA-tracrRNA-crRNA-tRNA-tracrRNA-crRNA-tRNA-tracrRNA-crRNA-tRNA-tracrRNA-crRNA, and the like).

In some embodiments, a guide RNA expressed from a synthetic snRNA promoter (or functional fragment or variant thereof) as described herein may further comprise an aptamer sequence (e.g., an MS2 aptamer). In some embodiments, the aptamer sequence recruits a deaminase. In some embodiments, the aptamer sequence recruits a reverse transcriptase. In some embodiments, a guide RNA may comprise one or to two or more aptamers (e.g., 1, 2, 3, 4, 5, 6, 7, 8, 9, 10 or more aptamers).

In some embodiments, a guide RNA expressed from a synthetic snRNA promoter (or functional fragment or variant thereof) as described herein may further comprise an RNA template for a reverse transcriptase. In some embodiments, a synthetic snRNA promoter (or functional fragment or variant thereof) as described herein is operably linked to a prime or REDRAW editing guide RNA (“PEgRNA” or “tagRNA”).

Cas9 is a class 2 CRISPR effector protein. Class 2 CRISPR-Cas systems rely on a single-component effector protein such as Cas9, in which dgRNA-or sgRNA-bound Cas protein recognizes and cleaves the target sequences. Cas9 recognizes a G-rich protospacer-adjacent motif (PAM) that is 3′ to its guide RNA binding site. In some embodiments, a CRISPR Cas9 protein can be a Cas9 protein from, for example, Streptococcus spp. (e.g., S. pyogenes, S. thermophilus), Lactobacillus spp., Bifidobacterium spp., Kandleria spp., Leuconostoc spp., Oenococcus spp., Pediococcus spp., Weissella spp., and/or Olsenella spp. Additional families of class 2 Cas effector proteins have been discovered: Cpf1 (also known as Cas12a), C2c1, CasX, and CasY (Burstein et al., Nature, 542:237-241, 2017).

Cas12a belongs to the class 2, Type V CRISPR system and utilizes a single RNA-guided endonuclease lacking tracrRNA. Cas12a systems recognize a T-rich protospacer-adjacent motif (PAM). The T-rich PAM allows for applications in genome editing in organisms with particularly AT-rich genomes or areas of interest with AT enrichment. The CRISPR array is processed into short mature crRNAs of 42-44 nucleotides in length. Each mature crRNA begins with 19 nucleotides of the direct repeat scaffold followed by 23-25 nucleotides of the spacer sequence. This crRNA arrangement contrasts with that of type II CRISPR-Cas systems in which the mature crRNA starts with 20-24 nucleotides of spacer sequence followed by approximately 22 nucleotides of direct repeat scaffold (Zetsche et al., Cell 163:759-771, 2015). Cas12a generates staggered cuts when cleaving a double-stranded DNA molecule which, is in contrast to a blunt-end cut (such as those generated by Cas9). An example of a Cas12a coding sequences comprising transit peptides for delivery to the nucleus of the cell is presented as SEQ ID NO:20 and encodes the protein presented as SEQ ID NO:22.

A CRISPR-Cas nuclease useful with this disclosure can include, but is not limited, to Cas9, C2c1, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), Csf5 and/or Mad7 nuclease. In some embodiments, the CRISPR-Cas nuclease may be a Cas9, Cas12a (also known as Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas 14c effector protein. In some embodiments, the CRISPR-Cas nuclease may be a Cas12a (also known as Cpf1) effector protein In some embodiments, a CRISPR-Cas nuclease useful with the disclosure may comprise a mutation in its nuclease active site (e.g., RuvC, HNH, e.g., RuvC site of a Cas12a nuclease domain; e.g., RuvC site and/or HNH site of a Cas9 nuclease domain). As described herein below, a CRISPR-Cas nuclease having a mutation in its nuclease active site, and therefore, no longer comprising nuclease activity, is commonly referred to as “dead,” e.g., dCas such as dCas9 or dCas12a. In some embodiments, a CRISPR-Cas nuclease domain or polypeptide having a mutation in its nuclease active site may have impaired activity or reduced activity as compared to the same CRISPR-Cas nuclease without the mutation, e.g., a nickase, e.g, Cas9 nickase, Cas12a nickase. Recently, CRISPR associated transposases (CAST) have been discovered and characterized. CASTs are comprised of Tn7-like transposase subunits, tnsB, tnsC, and tniQ, and the Type V-K CRISPR effector, Cas12k, catalyzes site-directed DNA transposition. Cas12k forms a complex with partially complementary non-coding RNA species, crRNA and tracrRNA and the tripartite ribonucleo-protein (RNP) complex recognizes chromosomal sites for transposition based on the presence of a protospacer adjacent motif (PAM) and complementarity between the variable portion of crRNA and the target DNA. The associated transposases, tnsB, tnsC and tniQ recognize the transposon by the conserved ‘left end’ (LE) and ‘right end’ (RE) boundaries and they insert it into a chromosomal site near the target sequence recognized by Cas12k, preferentially between a TA dinucleotide. Two homologous CAST systems, native in the cyanobacteria species Scytonema hofmanni (UTEX B 2349) and Anabaena cylindrica (PCC 7122) have been demonstrated to be functional for transposition in E. coli (Strecker et al., Science 365 (6448): 48-53, 2019).

Expression Strategies for gRNAs

The disclosure provides, in certain embodiments, novel combinations of synthetic snRNA promoters (and functional fragments and variants thereof) and a DNA sequence encoding one or more guide nucleic acid molecules. Guide nucleic acid molecules provided herein can be DNA, RNA, or a combination of DNA and RNA.

In one embodiment, a synthetic snRNA promoter (or functional fragment or variant thereof) is operably linked to one or more gRNA-encoding sequences, in order to constitutively express the gRNA(s) in transformed cells. This may be desirable, for example in some embodiments, when the resulting gRNA transcripts are retained in the nucleus and will thus be optimally located within the cell to guide nuclear processes. This may also be desirable, for example in some embodiments, when the activity of the CRISPR system is low or the frequency of finding and cleaving the target site is low. It may also be desirable in some embodiments when a promoter for a specific cell type, such as the germ line, is not known for a given species of interest.

In another embodiment, a fragment of the synthetic snRNA promoter (or functional fragment or variant thereof) comprising the necessary cis-regulatory elements to drive transcription can be used to express one or more gRNAs. The disclosed full length synthetic snRNA promoters, presented as SEQ ID NOs: 1-16 are each 300 bp in length. Recombinant DNA constructs (also referred to as “constructs” or “expression constructs”) comprising multiple synthetic snRNA promoters may become large as additional expression cassettes are cloned in tandem. This may result in issues affecting stability and transformation. Therefore, in certain instances the synthetic snRNA promoters may be truncated to reduce the size of the construct, so long as the truncated synthetic snRNA promoter retains the ability to drive transcription of the gRNA.

Multiple synthetic snRNA promoters (or functional fragments or variants thereof) with differing sequences may be utilized to minimize problems in construct stability, which is typically associated with sequence repeats and may be utilized to facilitate stacking of multiple gRNA cassettes in the same transformation construct.

In some embodiments, a synthetic snRNA promoter (or functional fragments or variants thereof) as described herein may drive the expression of a single gRNA. In some embodiments, a synthetic snRNA promoter (or functional fragments or variants thereof) as described herein may drive the expression of arrays of 2, 3, 4, 5, 6, 7, 8, 9, 10, 11, 12, 13, 14, 15, 16, 17, 18, 19, 20, 21, 22, 23, 24, 25, 26, 27, 28, 29, 30 or more gRNAs. Each individual guide sequence may target the same target sequence or different target sequences. This configuration is suitable for multiplex genetic manipulation (e.g., targeting multiple genes). Several strategies have been described in the art to facilitate the processing of individual crRNAs from a single transcript and it is known that the Cas12a effector protein can directly process individual crRNAs from a single transcript of a crRNA array (also referred to as guide RNA array). In some embodiments, a synthetic snRNA promoter (or functional fragments or variants thereof) as described herein may be used to drive gRNA arrays where the expression cassette comprises at least two or more gRNAs (i.e., gRNA units) separated by one or more tRNA cleavage sequence (as described in WO 2016/061481). A RNA cleavage sequence includes any sequence and/or structural motif that actively interacts with and is cleaved by a cell's endogenous tRNA system such as RNase P, RNase Z and RNase E (bacteria). This can include structural recognition elements such as the acceptor stem, D-loop arm, T Psi C loop as well as specific sequence motifs. In another embodiment, a synthetic snRNA promoter (or functional fragments or variants thereof) as described herein may be used to drive gRNA arrays comprising two or more gRNAs separated by one or more ribozyme cleavage sites (Tang et al., Mol. Plant 9:1088-1091, 2016). In another embodiment, a synthetic snRNA promoter (or functional fragments or variants thereof) as described herein may be used to drive gRNA arrays comprising two or more gRNAs arrays separated by one or more Csy4 ribonuclease recognition sites (Tsai et al., Nat. Biotechnol, 32 (6): 569-576, 2014).

In some embodiments, a synthetic snRNA promoter (or functional fragments or variants thereof) as described herein may be used to drive the expression of a prime or REDRAW editing gRNA (PEgRNA or tagRNA). Prime editing is a genome editing method that directly writes new genetic information into a targeted DNA site using a nucleic acid programmable DNA binding protein (napDNAbp) (e.g., Cas9) working in association with a polymerase (e.g., in the form of a fusion protein or otherwise provided in trans with the napDNAbp), wherein the prime editing system is programmed with a specialized prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA) (WO 2020/191248). In some embodiments, a synthetic snRNA promoter (or functional fragments or variants thereof) as described herein is used to drive expression of a PEgRNA that comprises a guide RNA and at least one nucleic acid extension arm comprising a DNA synthesis template wherein the nucleic acid extension arm is positioned at the 3′ or 5′ end of the guide RNA.

In another embodiment, a synthetic snRNA promoter (or functional fragments or variants thereof) as described herein may be used to drive the expression of an augmented gRNA that further comprises an RNA mobility sequence that enables cell to cell movement of the RNA. The RNA mobility sequence may be a sequence derived from plant genes like the Flowering Time (FT) gene, BEL5, GAI, tRNA-like motif, or LeT6 (WO 2021/041001).

In other embodiments, a synthetic snRNA promoter (or functional fragments or variants thereof) as described herein may be used to drive the expression of CRISPR RNA (crRNA), a mature crRNA, a precursor crRNA, a crRNA fragment, a trans-activating crRNA (tracrRNA) or a tracrRNA fragment.

In some embodiments, a synthetic snRNA promoter (or functional fragments or variants thereof) as described herein may be used to drive the expression of gRNAs that are compatible with other forms of CRISPR-mediated gene editing, such as base editing (Komor et al., Nature 533, 420-424, 2016; Gaudelli et. al., Nature 551:464-471, 2017; Komor et. al., Science Advances Vol 3: No. 8, 2017; and Rees et. al., Nat Rev Genet. 19 (12): 770-788, 2018).

In some embodiments, a synthetic snRNA promoter (or functional fragments or variants thereof) as described herein may be used to drive the expression of gRNAs that are compatible with the CRISPR Associated Transposase systems (CAST) such as those derived from Scytonema hofmanni (ShCAST) and Anabaena cylindrica (AcCAST) (Strecker et al., Science 365 (6448): 48-53, 2019).

In some embodiments, a synthetic snRNA promoter (or functional fragments or variants thereof) as described herein may be used to drive the expression of one or more non-protein-coding RNAs (npcRNAs). Non-limiting examples of non-protein-coding RNAs include microRNAs (miRNAs), miRNA precursors, small interfering RNAs (siRNAs), small RNAs (22-26 nt in length) and precursors encoding same, heterochromatic siRNAs (hc-siRNAs), Piwi-interacting RNAs (piRNAs), hairpin double stranded RNAs (hairpin dsRNAs), trans-acting siRNAs (ta-siRNAs), naturally occurring antisense siRNAs (nat-siRNAs) and tRNAs, and/or guide RNAs (gRNAs).

### Expression Strategies for CRISPR Class 2, Type II or Type V-Associated Genes

The disclosure provides novel synthetic snRNA promoters (and functional fragments and variants thereof) for use in sequence-specific CRISPR-mediated cleavage for molecular breeding by providing transcription of, for example, a gRNA comprising a spacer sequence used to target a site for endonuclease cleavage by at least one Cas protein. In certain embodiments, the target site is a genomic target site. In some embodiments, the genomic target site is native or transgenic. In addition, CRISPR systems can be customized to catalyze cleavage at one or more genomic target sites.

One aspect of this disclosure is to introduce into a plant cell an expression construct comprising one or more cassettes encoding a synthetic snRNA promoter (or a functional fragment or variant thereof) as described herein, operably linked to a nucleotide sequence encoding one or more gRNAs, including a copy of a spacer sequence complementary to a target site (e.g., a genomic target site), and an expression construct encoding a Type I, Type II, Type III, Type IV, Type V, or Type VI CRISPR associated protein to modify the plant cell in such a way that the plant cell, or a plant comprised of such cells, will subsequently exhibit a beneficial trait. In one non-limiting example, the trait is a trait such as improved yield, resistance to biotic or abiotic stress, herbicide tolerance, insect tolerance, or other improvements in agronomic performance. The ability to generate such a plant cell derived therefrom depends on introducing the CRISPR system using transformation constructs and cassettes described herein.

The expression construct encoding a CRISPR associated protein may comprise a promoter. In certain embodiments, the promoter is a constitutive promoter, a tissue specific promoter, a developmentally regulated promoter, or a cell cycle regulated promoter. Certain contemplated promoters include ones that only express in the germline or reproductive cells, among others. Such developmentally regulated promoters have the advantage of limiting the activity of the CRISPR system to only those cells in which the CRISPR associated protein is expressed. In some embodiments, a CRISPR-mediated genetic modification (e.g., chromosomal or episomal dsDNA cleavage) is limited only to cells that are involved in transmitting their genome from one generation to the next. This might be useful if broader expression of the CRISPR system were genotoxic or had other unwanted effects. Examples of such promoters include the promoters of genes encoding DNA ligases, recombinases, replicases, and so on.

In some embodiments, a recombinant DNA construct as described herein contains one or more synthetic snRNA promoters, or fragments or variants thereof, that express high levels of a DNA sequence encoding one or more gRNAs. A recombinant DNA construct that expresses a gRNA that guides a CRISPR class 2, type II or type V-associated protein with endonuclease activity to a specific genomic sequence, such that the specific genomic sequence is cleaved and produces a double-stranded break which is repaired by a double strand break repair pathway, which may include, for example, non-homologous end-joining, microhomology mediated end joining (MMEJ) homologous recombination, synthesis-dependent strand annealing (SDSA), single-strand annealing (SSA), or a combination thereof thereby disrupting the native locus, may be particularly useful.

In one embodiment, a CRISPR system comprises at least one Type I, Type II, Type III, Type IV, Type V, or Type VI CRISPR-associated protein and one gRNA comprising a copy of a spacer sequence complementary to an endogenous target site.

In some embodiments, the CRISPR system can include catalytically inactive CRISPR endonucleases. Such an endonuclease would comprise a domain that retains the ability to bind its target nucleic acid but has a diminished, or eliminated, ability to cleave a nucleic acid molecule, as compared to a control nuclease. In some embodiments, the catalytically inactive nuclease is a catalytically inactive Cas9. In some embodiments, the catalytically inactive Cas9 produces a nick in one of the target DNA strands. In some embodiments, the catalytically inactive Cas9, known as dead Cas9 (dCas9), lacks all nuclease activity. In some embodiments, the catalytically inactive nuclease is a catalytically inactive Cas12a. In some embodiments, the catalytically inactive Cas12a produces a nick in one of the target DNA strands. In some embodiments, the catalytically inactive Cas12a, known as dead Cas12a (dCas12a), lacks all DNase activity.

The present disclosure also provides for use of CRISPR-mediated double-stranded DNA cleavage to genetically alter expression and/or activity of a gene or gene product of interest in a tissue- or cell-type specific manner to improve productivity or provide another beneficial trait, wherein the nucleic acid of interest may be endogenous or transgenic in nature. Thus, in one embodiment, a CRISPR system is engineered to mediate disruption at specific sites in a gene of interest. Genes of interest include those for which altered expression level/protein activity is desired. These DNA cleavage events can be either in coding sequences or in regulatory elements within the gene.

This disclosure provides for the introduction of components of a CRISPR system (e.g., a CRISPR-associated protein and its cognate gRNA) into a cell. Examples of CRISPR-associated proteins include natural and engineered (e.g., modified, including codon-redesigned) nucleotide sequences encoding polypeptides with nuclease activity such as Cas9 from Streptococcus pyogenes, Streptococcus thermophilus, or Bradyrhizobium sp.; Cpf1 (also known as Cas12a) from Francisella novicida (FnCpf1), Prevotella sp., Acidaminococcus sp. BV3L6, and Lachnospiraceae bacterium ND2006 (LbCpf1); C2c1 from Alicyclobacillus acidoterrestris, Bacilli sp., Verrucomicrobia sp., α-proteobacteria, or δ-proteobacteria; CasX from Planctomycetes and δ-proteobacteria; or CasY from Candidatus Kerfeldbacteria, Candidatus Vogelbacteria, Candidatus Parcubacteria, or Candidatus komeilibacteria.

In particular embodiments, the codon-redesigned FnCas12a and LbCas12a nucleotide sequences and expression cassettes include recombinant nucleic acid sequences disclosed in U.S. 2020/0080096, the contents and disclosures of which are incorporated herein by reference.

The catalytically active CRISPR-associated gene (e.g., Cas9 endonuclease, Cas12b endonuclease, CasX endonuclease, CasY endonuclease, or Cpf1 endonuclease) can be introduced into, or produced by, a target cell. Various methods may be used to carry this out, as disclosed herein.

### Transient Expression of CRISPRs

In some embodiments, one or more expression cassettes encoding the gRNA and/or CRISPR associated protein components of a Type I, Type II, Type III, Type IV, Type V, or Type VI CRISPR-Cas system is transiently introduced into a cell. In certain embodiments, the introduced one or more expression cassettes encoding the gRNA and/or CRISPR associated protein is provided in sufficient quantity to modify the cell but does not persist after a contemplated period of time has passed or after one or more cell divisions. In such embodiments, no further steps are needed to remove or segregate the one or more expression cassettes encoding the gRNA and/or CRISPR associated protein from the modified cell. In yet other embodiments of this disclosure, double-stranded DNA fragments are also transiently introduced into a cell along with one or more expression cassettes encoding the gRNA and/or CRISPR associated protein.

In another embodiment, mRNA encoding the CRISPR associated protein is introduced into a cell. In such embodiments, the mRNA is translated to produce the CRISPR associated protein in sufficient quantity to modify the cell (in the presence of at least one gRNA whose expression is driven by a synthetic snRNA promoter (or functional fragments or variants thereof) as described herein) but does not persist after a contemplated period of time has passed or after one or more cell divisions. In such embodiments, no further steps are needed to remove or segregate the CRISPR associated protein from the modified cell.

In one embodiment of this disclosure, a catalytically active CRISPR associated protein is prepared in vitro prior to introduction to a plant cell comprising at least one gRNA whose expression is driven by a synthetic snRNA promoter (or functional fragment or variant thereof) as described herein. The method of preparing a CRISPR associated protein depends on its type and properties and would be known by one of skill in the art. For example, if the CRISPR associated protein is a large and monomeric protein, the active form of the CRISPR associated protein can be produced via bacterial expression, in vitro translation, via yeast cells, in insect cells, or by other protein production techniques known in the art. After expression, the CRISPR associated protein is isolated, refolded if needed, purified and optionally treated to remove any purification tags, such as a His-tag. Once crude, partially purified, or more completely purified CRISPR associated proteins are obtained, the protein may be introduced to, for example, a plant cell via electroporation, by bombardment with CRISPR associated protein coated particles, by chemical transfection or by some other means of transport across a cell membrane. Methods for introducing proteins and nucleic acids into plant cells are well known in the art. The protein can also be delivered using nanoparticles, which can deliver a combination of active protein and nucleic acid. Once a sufficient quantity of the CRISPR-associated protein is introduced so that an effective amount of in vivo activity is present, along with the appropriate gRNA, the target sequences within the genome are cleaved. It is also recognized that one skilled in the art might create a CRISPR associated protein that is inactive but is activated in vivo by native processing machinery; such a CRISPR associated protein is also contemplated by this disclosure.

In another embodiment, a construct that will transiently express a gRNA and/or CRISPR associated protein is created and introduced into a plant cell. In yet another embodiment, the construct will produce sufficient quantities of the gRNAs and/or CRISPR associated protein in order for the desired episomal or genomic target site or sites to be effectively modified. For instance, the disclosure contemplates preparation of a construct that can be bombarded, electroporated, chemically transfected or transported by some other means into a plant cell. Such a construct could have several useful properties. For instance, in one embodiment, the construct can replicate in a bacterial host such that the construct can be produced and purified in sufficient quantities for transient expression. In another embodiment, the construct can encode a herbicide resistance gene to allow selection for the construct in a host, or the construct can also comprise an expression cassette to provide for the expression of the gRNA and/or CRISPR associated protein in a plant. In a further embodiment, the CRISPR associated protein expression cassette could contain a promoter region, a 5′ untranslated region, an optional intron to aid expression, a multiple cloning site to allow facile introduction of a DNA sequence encoding the CRISPR associated protein, and a 3′ UTR. In particular embodiments, the promoter of the CRISPR associated protein expression cassette could be a constitutive promoter, tissue specific promoter, or other type of promoter that expresses in a plant cell. In a further embodiment, the gRNA expression cassette could contain a snRNA promoter (or functional fragment or variant thereof) as described herein, a gRNA encoding sequence, and a short poly-T region that terminates transcription. In some embodiments, the promoters in the gRNA expression cassettes would be synthetic snRNA promoters selected from any of SEQ ID NOs: 1-16. In some embodiments, it can be beneficial to include unique restriction sites at one or at each end of the expression cassette to allow the production and isolation of a linear expression cassette, which can then be free of other construct elements. The untranslated leader regions, in certain embodiments, can be plant-derived untranslated regions. Use of an intron, which can be plant-derived, is contemplated when the expression cassette is being transformed or transfected into a monocot or dicot cell.

In other embodiments, one or more elements in the construct include a spacer complementary to a target site contained within an episomal or genomic sequence. This facilitates CRISPR-mediated modification within the expression cassette, enabling removal and/or insertion of elements such as promoters and transgenes.

In another approach, a transient expression construct may be introduced into a plant cell using a bacterial or viral construct host. For example, Agrobacterium is one such bacterial construct that can be used to introduce a transient expression construct into a host plant cell. When using a bacterial, viral or other construct host system, the transient expression construct is contained within the host construct system. For example, if the Agrobacterium host system is used, the transient expression cassette would be flanked by one or more T-DNA borders and cloned into a binary construct. Many such construct systems have been identified in the art (reviewed in Hellens et al., 2000).

In embodiments, whereby one or more of the gRNA and/or CRISPR associated protein components of a CRISPR system is transiently introduced in sufficient quantities to modify a plant cell, a method of selecting the modified plant cell may be employed. In one such method, a second nucleic acid molecule containing a selectable marker is co-introduced with the transient gRNA and/or CRISPR associated protein. In this embodiment, the co-introduced marker may be part of a molecular strategy to introduce the marker at a target site. For example, the co-introduced marker may be used to disrupt a target gene by inserting between genomic target sites. In another embodiment, the co-introduced nucleic acid may be used to produce a visual marker protein such that transfected cells can be cell-sorted or isolated by some other means. In yet another embodiment, the co-introduced marker may randomly integrate or be directed via a second gRNA: CRISPR associated protein complex to integrate at a site independent of the primary genomic target site. In still yet another embodiment, the co-introduced molecule may be targeted to a specific locus via a double strand break repair pathway, which may include, for example, non-homologous end-joining (NHEJ), microhomology mediated end joining (MMEJ), homologous recombination, synthesis-dependent strand annealing (SDSA), single-strand annealing (SSA), or a combination thereof, at the genomic target site(s). In the above embodiments, the co-introduced marker may be used to identify or select for cells that have likely been exposed to the gRNA and/or CRISPR associated protein and therefore are likely to have been modified by the CRISPR.

### Stable Expression of CRISPRs

In another embodiment, one or more expression constructs encoding one or more components of a CRISPR system (e.g., a CRISPR associated protein and its cognate gRNA) are stably transformed into a plant cell. In this embodiment, the design of the transformation construct provides flexibility for when and under what conditions the gRNA and/or CRISPR associated protein is expressed. Furthermore, the transformation construct can be designed to comprise a selectable or visible marker that will provide a means to isolate or efficiently select cell lines that contain one or more expression constructs encoding one or more components of a CRISPR system and/or have been modified by the CRISPR system.

Cell transformation systems have been described in the art and descriptions include a variety of transformation constructs. For example, for plant transformations, two principal methods include Agrobacterium-mediated transformation and particle gun bombardment-mediated (e.g., biolistic) transformation. In both cases, the nucleotide sequences encoding the CRISPR system components are introduced via one or more expression cassettes. In a further embodiment, a CRISPR associated protein expression cassette could contain a promoter region, a 5′ untranslated region, an optional intron to aid expression, a multiple cloning site to allow facile introduction of a DNA sequence encoding a CRISPR associated protein, and a 3′ UTR. In particular embodiments, the promoter of the CRISPR associated protein expression cassette could be a constitutive promoter, a tissue specific promoter, a developmentally regulated promoter, a cell cycle regulated promoter, or a germline specific promoter. In a further embodiment, the gRNA expression cassette could contain a snRNA promoter (or functional fragment or variant thereof) as described herein, a gRNA encoding sequence, and a short poly-T region that terminates transcription. In particular embodiments, the promoter in a gRNA expression cassette would be a synthetic snRNA promoter selected from any of SEQ ID NOs: 1-16.

For particle bombardment or with protoplast transformation, the expression cassette can be an isolated linear fragment or may be part of a larger construct that might contain bacterial replication elements, bacterial selectable markers or other elements. The one or more gRNA and/or CRISPR associated protein expression cassette(s) may be physically linked to a marker cassette or may be mixed with a second nucleic acid molecule encoding a marker cassette. In some embodiments, a marker cassette is comprised of necessary elements to express a visual or selectable marker that allows for efficient selection of transformed cells. In the case of Agrobacterium-mediated transformation, the one or more expression cassettes may be adjacent to or between flanking T-DNA borders and contained within a binary construct. In another embodiment, the one or more expression cassettes may be outside of the T-DNA. The presence of the one or more expression cassettes in a cell may be manipulated by positive or negative selection regime(s). Furthermore, a selectable marker cassette may also be within or adjacent to the same T-DNA borders or may be somewhere else within a second T-DNA on the binary construct (e.g., a 2 T-DNA system).

In some embodiments, cells that have been modified by a CRISPR system, either transiently or stably, are carried forward along with unmodified cells. The cells can be sub-divided into independent clonally derived lines or can be used to regenerate independently derived plants. Individual plants or clonal populations regenerated from such cells can be used to generate independently derived lines. At any of these stages a molecular assay can be employed to screen for cells, plants or lines that have been modified. Cells, plants or lines that have been modified continue to be propagated and unmodified cells, plants or lines are discarded. In some embodiments, the presence of an active CRISPR system in a cell is essential to ensure the efficiency of the overall process.

### Transformation Methods

Methods for transforming or transfecting a cell are well known in the art. Methods for plant transformation using Agrobacterium or DNA coated particles are well known in the art and are incorporated herein. Suitable methods for transformation of host cells for use with the current disclosure are believed to include virtually any method by which DNA can be introduced into a cell, for example by Agrobacterium-mediated transformation (U.S. Pat. Nos. 5,563,055; 5,591,616; 5,693,512; 5,824,877; 5,981,840; and 6,384,301) and by acceleration of DNA coated particles (U.S. Pat. Nos. 5,015,580; 5,550,318; 5,538,880; 6,160,208; 6,399,861; and 6,403,865), etc. Through the application of techniques such as these, the cells of virtually any species may be stably transformed.

Various methods for selecting transformed cells have been described. For example, one might utilize a drug resistance marker such as a neomycin phosphotransferase protein to confer resistance to kanamycin or to use 5-enolpyruvyl shikimate phosphate synthase to confer tolerance to glyphosate. In another embodiment, a carotenoid synthase is used to create an orange pigment that can be visually identified. These three exemplary approaches can each be used effectively to isolate a cell or plant or tissue thereof that has been transformed and/or modified by a CRISPR system.

When a nucleic acid sequence encoding a selectable or screenable marker is inserted into a genomic target site, the marker can be used to detect the presence or absence of a CRISPR system or its activity. This may be useful once a cell has been modified by a CRISPR system, and recovery of a genetically modified cell that no longer contains the CRISPR expression cassette(s), or a regenerated plant from such a modified cell, is desired. In other embodiments, the marker may be intentionally designed to integrate at the genomic target site, such that it can be used to follow a modified cell independently of the CRISPR system. The marker can be a gene that provides a visually detectable phenotype, such as in the seed, to allow rapid identification of seeds that carry or lack a CRISPR expression cassette.

This disclosure provides for a means to regenerate a plant from a cell with a repaired double-stranded break within a genomic target site. The regenerate can then be used to propagate additional plants.

The disclosure additionally provides novel plant transformation constructs and expression cassettes which include synthetic snRNA promoters, and combinations thereof, with CRISPR-associated gene(s) and gRNA/expression cassettes. The disclosure further provides methods of obtaining a plant cell, a whole plant, and a seed or embryo that have been specifically modified using CRISPR-mediated cleavage. This disclosure also relates to a novel plant cell containing a CRISPR-associated Cas endonuclease expression construct and gRNA expression cassettes.

### Targeting Using Blunt-End Oligonucleotides

In certain embodiments, a CRISPR system (e.g., a CRISPR/Cas9 system or CRISPR/Cas12a system) can be utilized for targeting 5′ insertion of a blunt-end double-stranded DNA fragment into a genomic target site of interest. In some embodiments, CRISPR-mediated endonuclease activity can introduce a double stand break (DSB) in the selected genomic target site and DNA repair, such as microhomology-driven non-homologous end-joining DNA repair, results in insertion of the blunt-end double-stranded DNA fragment into the DSB. In some embodiments, blunt-end double-stranded DNA fragments can be designed with 1-10 bp of microhomology, on both the 5′ and 3′ ends of the DNA fragment, that correspond to the 5′ and 3′ flanking sequence at the cut site in the genomic target site.

### Use of CRISPR Systems in Molecular Breeding

In some embodiments, genome knowledge is utilized for targeted genetic alteration of a genome. At least one gRNA can be designed to target at least one region of a genome to alter that region of the genome. This aspect of the disclosure may be especially useful for genetic alterations. The resulting plant could have a modified phenotype or other property depending on the gene or genes that have been altered. Previously characterized mutant alleles or introduced transgenes can be targeted for CRISPR-mediated modification, enabling creation of improved mutants or transgenic lines.

In another embodiment, a gene targeted for deletion or disruption may be a transgene that was previously introduced into the target plant or plant cell. This has the advantage of allowing an improved version of a transgene to be introduced or by allowing disruption of a selectable marker encoding sequence. In yet another embodiment, a gene targeted for disruption via a CRISPR system is at least one transgene that was introduced on the same construct or expression cassette as (an) other transgene(s) of interest and resides at the same locus as another transgene. It is understood by those skilled in the art that this type of CRISPR-mediated modification may result in deletion or insertion of additional sequences. Thus, it may, in certain embodiments, be preferable to generate a plurality of plants or plant cells in which a deletion has occurred, and to screen such plants or plant cells using standard techniques to identify specific plants or plant cells that have minimal alterations in their genomes following CRISPR-mediated modification. Such screens may utilize genotypic and/or phenotypic information. In such embodiments, a specific transgene may be disrupted while leaving the remaining transgene(s) intact. This avoids having to create a new transgenic line containing the desired transgenes without the undesired transgene.

In another aspect, the present disclosure includes methods for inserting a DNA fragment of interest into a specific site of a plant's genome, wherein the DNA fragment of interest is from the genome of the plant or is heterologous with respect to the plant. This disclosure allows one to select or target a particular region of the genome for nucleic acid (e.g., transgene) stacking (e.g., mega-locus). A targeted region of the genome may thus display linkage of at least one transgene to a haplotype of interest associated with at least one phenotypic trait and may also result in the development of a linkage block to facilitate transgene stacking and transgenic trait integration, and/or development of a linkage block while also allowing for conventional trait integration.

### Use of CRISPR Systems in Trait Integration

Directed insertion, in at least one genomic target site, of DNA fragments of interest, via CRISPR-mediated cleavage allows for targeted integration of multiple nucleic acids of interest (e.g., a trait stack) to be added to the genome of a plant in either the same site or different sites. Sites for targeted integration can be selected based on knowledge of the underlying breeding value, transgene performance in that location, underlying recombination rate in that location, existing transgenes in that linkage block, or other factors. Once the stacked plant is assembled, it can be used as a trait donor for crosses to germplasm being advanced in a breeding pipeline or be directly advanced in the breeding pipeline.

The present disclosure includes methods for inserting at least one nucleic acid of interest into at least one site, wherein the nucleic acid of interest is from the genome of a plant, such as a QTL or allele, or is transgenic in origin. A targeted region of the genome may thus display linkage of at least one transgene to a haplotype of interest associated with at least one phenotypic trait (as described in U.S. Patent Application Publication No. 2006/0282911), development of a linkage block to facilitate transgene stacking and transgenic trait integration, development of a linkage block to facilitate QTL or haplotype stacking and conventional trait integration, etc.

In another embodiment of this disclosure, multiple unique gRNAs can be used to modify multiple loci within one linkage block contained on one chromosome by making use of knowledge of genomic sequence information and the ability to design custom gRNAs as described in the art. A gRNA that is specific for, or can be directed to, a genomic target site that is upstream of the locus containing the non-target allele is designed or engineered as necessary. A second gRNA that is specific for, or can be directed to, a genomic target site that is downstream of the target locus containing the non-target allele is also designed or engineered. The gRNAs may be designed such that they complement genomic regions where there is no homology to the non-target locus containing the target allele. Both gRNAs may be introduced into a cell using one of the methods described above.

The ability to execute targeted integration relies on the action of the gRNA: CRISPR associated protein. This advantage provides methods for engineering plants of interest, including a plant or cell, comprising at least one genomic modification.

A custom gRNA can be utilized in a CRISPR system to generate at least one trait donor to create a custom genomic modification event that is then crossed into at least one second plant of interest, including a plant, wherein CRISPR associated protein delivery can be coupled with the gRNA of interest to be used for genome editing. In other aspects one or more plants of interest are directly transformed with the CRISPR system and at least one double-stranded DNA fragment of interest for directed insertion. It is recognized that this method may be executed in various cell, tissue, and developmental types, including gametes of plants. It is further anticipated that one or more of the elements described herein may be combined with use of promoters specific to particular cells, tissues, plant parts and/or developmental stages, such as a meiosis-specific promoter.

In addition, the disclosure contemplates the targeting of a transgenic element already existing within a genome for deletion or disruption. This allows, for instance, an improved version of a transgene to be introduced, or allows selectable marker removal. In yet another embodiment, a gene targeted for disruption via CRISPR-mediated cleavage is at least one transgene that was introduced on the same construct or expression cassette as (an) other transgene(s) of interest and resides at the same locus as another transgene.

In one aspect, the disclosure provides a method for modifying a locus of interest in a cell comprising (a) identifying at least one locus of interest within a DNA sequence; (b) introducing into the cell an expression cassette comprising a synthetic snRNA promoter selected from any of SEQ ID NOs: 1-16 operably linked to a nucleotide sequence encoding a gRNA and an expression cassette comprising a plant expressible promoter operably linked to a nucleic acid sequence encoding a CRISPR associated protein, wherein the gRNA and/or CRISPR associated protein is expressed transiently or stably; (c) assaying the cell for a CRISPR-mediated modification in the DNA making up or flanking the locus of interest; and (d) identifying the cell or a progeny cell thereof as comprising a modification in said locus of interest.

Another aspect provides a method for modifying multiple loci of interest in a cell comprising (a) identifying multiple loci of interest within a genome; (b) introducing into at least one cell multiple expression cassettes, each comprising a synthetic snRNA promoter selected from any of SEQ ID NOs: 1-16 operably linked to a nucleotide sequence encoding a gRNA, wherein the synthetic snRNA promoters can be the same or distinct, and at least one expression cassette comprising a plant expressible promoter operably linked to a nucleic acid sequence encoding a CRISPR associated protein according to the disclosure, wherein the cell comprises the genomic target sites and the gRNAs and CRISPR associated protein are expressed transiently or stably and creates a modified locus, or loci, that includes at least one CRISPR-mediated cleavage event; (c) assaying the cell for CRISPR-mediated modifications in the DNA making up or flanking each locus of interest; and (d) identifying a cell or a progeny cell thereof which comprises a modified nucleotide sequence at said loci of interest.

The disclosure further contemplates sequential modification of a locus of interest, by two or more gRNAs and CRISPR associated protein(s) according to the disclosure. Genes or other sequences added by the action of such a first CRISPR-mediated genomic modification may be retained, further modified, or removed by the action of a second CRISPR-mediated genomic modification.

The present disclosure thus includes compositions and methods for modifying a locus of interest in a crop plant such as maize (corn: Zea mays subsp. mays); corn varieties (flour corn (Zea mays var. amylacea), popcorn (Zea mays var. everta), dent corn (Zea mays var. indentata), flint corn (Zea mays var. indurate), sweet corn (Zea mays var. saccharata and Zea mays var. rugose), waxy corn (Zea mays var. ceratina), amylomaize (Zea mays), pod corn (Zea mays var. tunicata Larrañaga ex A. St. Hil.), striped maize (Zea mays var. japonica); soybean (Glycine max); cotton (Gossypium hirsutum; Gossypium sp.); peanut (Arachis hypogaea); barley (Hordeum vulgare); oats (Avena sativa); orchard grass (Dactylis glomerata); rice (Oryza sativa, including indica and japonica varieties); sorghum (Sorghum bicolor); sugarcane (Saccharum sp.); tall fescue (Festuca arundinacea); turfgrass species (e.g. species: Agrostis stolonifera, Poa pratensis, Stenotaphrum secundatum); wheat (Triticum aestivum); alfalfa (Medicago sativa); members of the genus Brassica which include but are not limited to, canola (Brassica napus and Brassica rapa), members of the genus Brassica (e.g. species: bok choy (B. rapa subsp. chinensis), bomdong (Brassica rapa var. glabra), choy sum (Brassica rapa subsp. parachinensis), field mustard (Brassica rapa subsp. oleifera), komatsuna (Brassica rapa subsp. perviridis, napa cabbage (Brassica rapa subsp. pekinensis), rapini (Brassica rapa var. rapifera), tatsoi (Brassica rapa subsp. narinosa), turnip (Brassica rapa subsp. rapa), yellow sarson (Brassica rapa subsp. trilocularis), Chinese cabbage, turnip, rapini, komatsuna (Brassica rapa (syn. Brassica campestris)), Mallorca cabbage (Brassica balearica), Abyssinian mustard or Abyssinian cabbage, used to produce biodiesel (Brassica carinata), elongated mustard (Brassica elongata), Mediterranean cabbage (Brassica. fruticulosa), St Hilarion cabbage (Brassica hilarionis), Indian mustard, brown and leaf mustards, Sarepta mustard (Brassica juncea), rapeseed, canola, rutabaga (swede, swede turnip, Swedish turnip) (Brassica napus), broadbeaked mustard (Brassica narinosa), black mustard (Brassica nigra), kale, cabbage, collard greens, broccoli, cauliflower, kai-lan, Brussels sprouts, kohlrabi (Brassica oleracea), tender green, mustard spinach (Brassica perviridis), brown mustard (Brassica rupestris), seventop turnip (Brassica septiceps), Asian mustard (Brassica. tournefortii), broccoli (B. oleracea); pepper (e.g. species: black pepper, white and green pepper (Piper nigrum), cubeb (Piper cubeba), Indian long pepper (Piper longum), Indonesian long pepper (Piper retrofractum), Voatsiperifery (Piper borbonense), Ashanti pepper (Piper guineense), banana pepper, bell pepper, cayenne pepper, jalapeño, Florina pepper, (Capsicum annuum cultivars), chili pepper (cultivars of Capsicum annuum, Capsicum frutescens, Capsicum chinense, Capsicum pubescens, and Capsicum baccatum), and datil pepper (Capsicum chinense cultivar); bean plant species (e.g. broad bean or fava bean (Vicia faba), common bean; includes the pinto bean, kidney bean, black bean, Appaloosa bean as well as green beans, and many others (Phaseolus vulgaris), tepary bean (Phaseolus acutifolius), runner bean (Phaseolus coccineus), lima bean (Phaseolus lunatus), a.k.a. P. dumosus, recognized as a separate species in 1995 (Phaseolus polyanthus), moth bean (Vigna aconitifolia), adzuki bean (Vigna angularis), urad bean (Vigna mungo), mung bean (Vigna radiata), Bambara bean or ground-bean (Vigna subterranea), ricebean (Vigna umbellata), cowpea; also includes the black-eyed pea, yardlong bean and others (Vigna unguiculata), chickpea or garbanzo bean (Cicer arietinum), pea (Pisum sativum), Indian pea (Lathyrus sativus), tuberous pea (Lathyrus tuberosus), lentil (Lens culinaris), hyacinth bean (Lablab purpureus), winged bean (Psophocarpus tetragonolobus), pigeon pea (Cajanus cajan), velvet bean (Mucuna pruriens), guar (Cyamopsis tetragonoloba), jack bean (Canavalia ensiformis), sword bean (Canavalia gladiata), horse gram (Macrotyloma uniflorum), tarwi (Lupinus mutabilis), lupini bean (Lupinus albus); gourd family members (Cucurbitaceae; e.g. genera: squash, pumpkin, zucchini, some gourds (Cucurbita), calabash (Lagenaria), watermelon (Citrullus such as Citrullus lanatus and Citrullus colocynthis), cucumber (Cucumis sativus), various melons (Cucumis melo, Cucumis metuliferus); spinach (Spinacia oleracea); carrot (Daucus carota subsp. sativus); tomato (Solanum lycopersicum); onion (Allium cepa L.); radish (Raphanus raphanistrum subsp. sativus); potato (Solanum tuberosum); ornamental plants; and oilseed crops such as soybean, canola, oil seed rape, oil palm, sunflower, olive, corn, cottonseed, peanut, flaxseed, safflower, and coconut.

The targeted genome modification may comprise a modified linkage block, the linking of two or more QTLs, disrupting linkage of two or more QTLs, gene insertion, gene replacement, gene conversion, deleting or disrupting a gene, transgenic event selection, transgenic trait donor selection, transgene replacement, or targeted insertion of at least one nucleic acid of interest.

### DEFINITIONS

The definitions and methods provided define the present disclosure and guide those of ordinary skill in the art in the practice of the present disclosure. Unless otherwise noted, terms are to be understood according to conventional usage by those of ordinary skill in the relevant art. Definitions of common terms and methods in molecular biology may also be found in Clark et al., Molecular Biology, Third Edition, Academic Press, Elsevier Inc., 2019; Alberts et al., Molecular Biology of The Cell, 5th Edition, Garland Science Publishing, Inc.: New York, 2007; Rieger et al., Glossary of Genetics: Classical and Molecular, 5th edition, Springer-Verlag: New York, 1991; King et al., A Dictionary of Genetics, 6th ed., Oxford University Press: New York, 15 2247; and Lewin, Genes IX, Oxford University Press: New York, 2007. The nomenclature for DNA bases as set forth at 37 CFR § 1.822 is used and set forth in WIPO Standard ST.26 (2021), Annex I, Tables 1 and 3.

As used herein, the terms “synthetic nucleotide sequence”, “artificial nucleotide sequence”, “synthetic promoter” and “synthetic snRNA promoter” refer to a nucleotide sequence that is not known to occur in nature or that is not naturally occurring. The gene regulatory elements of the present disclosure comprise synthetic nucleotide sequences. The recombinant DNA molecules of the present disclosure comprise synthetic nucleotide sequences. Preferably, synthetic nucleotide sequences share little or no extended homology to natural sequences. Extended homology in this context generally refers to 100% sequence identity extending beyond about 25 nucleotides of contiguous sequence.

Reference in this application to an “isolated DNA molecule,” or an equivalent term or phrase, is intended to mean that the DNA molecule is one that is present alone or in combination with other compositions, but not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element is not within the genome of the organism and at the location within the genome in which it is naturally found. In one embodiment, the term “isolated” refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state. For the purposes of this disclosure, any transgenic nucleotide sequence, i.e., the nucleotide sequence of the DNA inserted into the genome of the cells of a plant or bacterium, or present in an extrachromosomal construct, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

As used herein, the term “heterologous” refers to the combination of two or more DNA sequences, RNA sequences, amino acid sequences, or protein domains when such a combination is not normally found in nature or when such a combination is provided in an orientation or order that is different than that found in nature. For example, the two DNA sequences may be derived from different species or created synthetically and/or the two DNA sequences may be derived from different genes, e.g., different genes from the same species or the same genes from different species. In one example, a regulatory element may be heterologous with respect to an operably linked transcribable DNA molecule if such a combination is not normally found in nature, e.g., the transcribable DNA molecule does not naturally occur operably linked to the regulatory element. In one embodiment, such a heterologous combination may comprise a regulatory element of any of SEQ ID NOs: 1-16 that may be chemically synthesized and may be operably linked to a transcribable DNA molecule, such as a DNA sequence encoding a non-protein-coding small RNA (npcRNA), for example a gRNA. In addition, a particular sequence can be “heterologous” with respect to a cell or organism into which it is introduced (for example, a sequence that does not naturally occur in that particular cell or organism). By “heterologous transcribable DNA molecule,” it is meant that a transcribable DNA molecule is heterologous with respect to a DNA sequence to which it is operably linked.

As used herein, the term “operably linked” refers to a first DNA molecule joined to a second DNA molecule, wherein the first and second DNA molecules are so arranged that the first DNA molecule affects the function of the second DNA molecule. The two DNA molecules may or may not be part of a single contiguous DNA molecule and may or may not be adjacent. For example, a promoter is operably linked to a DNA molecule if the promoter modulates transcription of the DNA molecule of interest in a cell. A leader, for example, is operably linked to a DNA sequence when it is capable of affecting the transcription or translation of the DNA sequence.

As used herein, a “recombinant” DNA molecule, polypeptide, protein, cell, or organism may be a non-naturally occurring or man-made creation using the tools of genetic engineering and as such is the product of human activity and would not otherwise normally occur in nature. A “recombinant DNA molecule” refers to a DNA molecule comprising a combination of DNA sequences or polynucleotides that would not naturally occur together without human intervention. For instance, a recombinant DNA molecule may be a DNA molecule that is comprised of at least two DNA molecule heterologous with respect to each other, a DNA molecule that comprises a DNA sequence that deviates from DNA sequences that exist in nature, or a DNA molecule that has been incorporated into a host cell's DNA by genetic transformation. In one embodiment, a recombinant DNA molecule as described herein is a DNA molecule comprising any one of SEQ ID NOs: 1-16 operably linked to at least one transcribable DNA molecule, for instance, where the transcribable DNA molecule is heterologous to the DNA molecule of any one of SEQ ID NOs: 1-16.

As used herein, the term “recombinant DNA construct” means any recombinant DNA molecule such as a plasmid, cosmid, virus, phage, or linear or circular DNA or RNA molecule, derived from any source, capable of genomic integration or autonomous replication, comprising a DNA molecule where at least one DNA molecule has been linked to another DNA molecule in a functionally operative manner, i.e., operably linked. As used herein, the term “construct” means any construct that may be used for the purpose of transformation, i.e., the introduction of heterologous DNA or RNA into a host cell. A construct typically includes one or more expression cassettes. In one embodiment, a recombinant DNA construct as described herein is a recombinant DNA molecule comprising at least one expression cassette. As used herein, an “expression cassette” refers to a recombinant DNA molecule comprising at least one transcribable DNA molecule operably linked to one or more regulatory elements, typically at least a promoter and a 3′ UTR. In one embodiment, the at least one transcribable DNA molecule of an expression cassette is a transgene. In another embodiment, the at least one transcribable DNA molecule of an expression cassette is a DNA sequence encoding a non-protein-coding small RNA (npcRNA), for example a gRNA. A recombinant DNA construct can be a linear fragment of DNA, a plasmid vector, or a binary plant transformation plasmid vector that is used to carry exogenous genetic material into a prokaryotic or eukaryotic cell.

As used herein, a “cis-regulatory elements” are regions of non-coding DNA which regulate the transcription of neighboring genes.

As used herein, the term “sequence identity” refers to the extent to which two optimally aligned polynucleotide sequences or two optimally aligned polypeptide sequences are identical. An optimal sequence alignment is created by manually aligning two sequences, e.g., a reference sequence and another sequence, to maximize the number of nucleotide matches in the sequence alignment with appropriate internal nucleotide insertions, deletions, or gaps. As used herein, the term “reference sequence” refers to a DNA sequence provided as any of SEQ ID NOs: 1-16.

As used herein, the terms “percent sequence identity”, “% sequence identity”, “percent identity”, and “% identity” refer to the identity fraction multiplied by 100. The “identity fraction” for a sequence optimally aligned with a reference sequence is the number of nucleotide matches in the optimal alignment, divided by the total number of nucleotides in the reference sequence, e.g., the total number of nucleotides in the full length of the entire reference sequence. Thus, one embodiment of the present disclosure provides a DNA molecule comprising a sequence that, when optimally aligned to a reference sequence, provided herein as any of SEQ ID NOs: 1-16, has at least about 85 percent identity, at least about 86 percent identity, at least about 87 percent identity, at least about 88 percent identity, at least about 89 percent identity, at least about 90 percent identity, at least about 91 percent identity, at least about 92 percent identity, at least about 93 percent identity, at least about 94 percent identity, at least about 95 percent identity, at least about 96 percent identity, at least about 97 percent identity, at least about 98 percent identity, at least about 99 percent identity, or at least about 100 percent identity to the reference sequence. In still further specific embodiments, a sequence having a percent identity to any of SEQ ID NOs: 1-16 may be defined as exhibiting promoter activity possessed by the starting sequence from which it is derived. A sequence having a percent identity to any of SEQ ID NOs: 1-16 may further comprise a “minimal promoter” which provides a basal level of transcription and is comprised of a TATA box or equivalent sequence for recognition and binding of the RNA polymerase III complex for initiation of transcription. In accordance with the disclosure, a promoter, promoter variant, or promoter fragment may be analyzed for the presence of known promoter elements, i.e., DNA sequence characteristics, such as a TATA box and other known transcription factor binding site motifs. Identification of such known promoter elements may be used by one of skill in the art to design variants of the promoter having a similar expression pattern to the original promoter.

The term “genome” encompasses not only chromosomal DNA found within the nucleus, but organelle DNA found within subcellular components (e.g., mitochondria, or plastid) of the cell.

As used herein, the term “genome editing” or “editing” refers to any modification of a nucleotide sequence in a site-specific manner. In the present disclosure genome editing techniques include the use of endonucleases, recombinases, transposases, helicases and any combination thereof. In an aspect, a “modification” comprises the hydrolytic deamination of cytidine or deoxycytidine to uridine or deoxyuridine, respectively. In some embodiments, a sequence-specific editing system comprises an adenine deaminase. In an aspect, a “modification” comprises the hydrolytic deamination of adenine or adenosine. In an aspect, a “modification” comprises the hydrolytic deamination of adenosine or deoxyadenosine to inosine or deoxyinosine, respectively. In an aspect, a “modification” comprises the insertion of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In another aspect, a “modification” comprises the deletion of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In a further aspect, a “modification” comprises the inversion of at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In still another aspect, a “modification” comprises the substitution of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In still another aspect, a “modification” comprises the duplication of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In some embodiments, a “modification” comprises the substitution of an “A” for a “C”, “G” or “T” in a nucleic acid sequence. In some embodiments, a “modification” comprises the substitution of a “C” for an “A”, “G” or “T” in a nucleic acid sequence. In some embodiments, a “modification” comprises the substitution of a “G” for an “A”, “C” or “T” in a nucleic acid sequence. In some embodiments, a “modification” comprises the substitution of a “T” for an “A”, “C” or “G” in a nucleic acid sequence. In some embodiments, a “modification” comprises the substitution of a “C” for a “U” in a nucleic acid sequence. In some embodiments, a “modification” comprises the substitution of a “G” for an “A” in a nucleic acid sequence. In some embodiments, a “modification” comprises the substitution of an “A” for a “G” in a nucleic acid sequence. In some embodiments, a “modification” comprises the substitution of a “T” for a “C” in a nucleic acid sequence.

As used herein, the term “target site” refers to a nucleotide sequence against which a gRNA/CRISPR associated protein system binds and/or exerts activity (e.g., a protospacer and a protospacer adjacent motif (PAM)) located in a DNA sequence that is selected for targeted modification. A target site may be genic or non-genic. A target site may be on a chromosome, episome, a locus, or any other DNA molecule in the genome (including chromosomal, chloroplastic, mitochondrial DNA, plasmid DNA) of a cell. The target site can be an endogenous site in the genome of a cell, or alternatively, the target site can be heterologous to the cell and thereby not be naturally occurring in the genome of the cell, or the target site can be found in a heterologous genomic location compared to where it occurs in nature.

As used herein, the term “genomic target site” refers to a target site (e.g., a protospacer and a protospacer adjacent motif (PAM)) located in a host genome selected for targeted modification.

As used herein, the term “protospacer” refers to a short DNA sequence (12 to 40 bp) that can be targeted by a CRISPR system guided by complementary base-pairing with the spacer sequence in the gRNA.

As used herein, the term “microhomology” refers to the presence of the same short sequence (1 to 10 bp) of bases in different polynucleotide molecules.

As used herein, the term “codon-optimized” refers to a polynucleotide sequence that has been modified to exploit the codon usage bias of a particular plant. The modified polynucleotide sequence still encodes the same, or substantially similar polypeptide as the original sequence but uses codon nucleotide triplets that are found in greater frequency in a particular plant.

As used herein, the term “non-protein-coding RNA” (npcRNA) refers to a non-coding RNA (ncRNA) which is a precursor small non-protein-coding RNA, or a fully processed non-protein-coding RNA, which are functional RNA molecules that are not translated into a protein.

A promoter is useful as a regulatory element for modulating the expression of an operably linked transcribable DNA molecule. As used herein, the term “promoter” refers generally to a nucleic acid sequence (that is, a DNA molecule) located upstream or 5′ to a transcriptional start codon and that is involved in recognition and binding of RNA polymerase, e.g., RNA polymerase II or RNA polymerase III, and other proteins, such as trans-acting transcription factors, to initiate transcription. A promoter may be initially isolated from the 5′ untranslated region (5′ UTR) of a genomic copy of a gene. In some embodiments, a promoter is operably linked 5′ to a leader sequence. Promoters may be synthetically produced or manipulated DNA molecules. Promoters may also be chimeric. Chimeric promoters are produced through the fusion of two or more heterologous DNA molecules. In some embodiments, the claimed DNA molecules and any fragments, or variants or derivatives thereof as described herein, are further defined as comprising promoter activity, i.e., are capable of acting as a promoter in a host cell, such as in a transgenic plant cell. In still further specific embodiments, a functional fragment may be defined as exhibiting promoter activity possessed by the starting promoter molecule from which it is derived, or a functional fragment may comprise a “minimal promoter” which provides a basal level of transcription and is comprised of a TATA box or equivalent DNA sequence for recognition and binding of the RNA polymerase II complex for initiation of transcription. A “plant promoter” is a native or non-native promoter that is functional in plant cells. A “snRNA promoter” is a native or non-native promoter that is functional in plant cells. Constitutive promoters are functional in most or all tissues of a plant throughout plant development. Tissue-, organ- or cell-specific promoters are expressed only or predominantly in a particular tissue, organ, or cell type, respectively. Rather than being expressed “specifically” in a given tissue, plant part, or cell type, a promoter may display “enhanced” expression, i.e., a higher level of expression, in one cell type, tissue, or plant part of the plant compared to other parts of the plant. Temporally regulated promoters are functional only or predominantly during certain periods of plant development or at certain times of day, as in the case of genes associated with circadian rhythm, for example. Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

In one embodiment, “functional fragments” or “fragments” of a promoter sequence disclosed herein are provided. Promoter fragments or functional fragments may comprise promoter activity, as described above, and may be useful alone or in combination with other promoters and promoter fragments, such as in constructing chimeric promoters, or in combination with other expression elements and expression element fragments. In some embodiments, fragments of a promoter are provided comprising at least about 50, at least about 75, at least about 95, at least about 100, at least about 125, at least about 150, at least about 175, at least about 200, at least about 225, at least about 250, at least about 275 contiguous nucleotides, or longer, of a DNA molecule having promoter activity as disclosed herein. According to another embodiment of the present disclosure, a fragment of any of SEQ ID NOs: 1-16 is provided that may have gene regulatory activity. In another embodiment, a fragment of any of SEQ ID NOs: 1-16 is provided that may comprise the same or similar gene regulatory activity as the reference sequence from which it is derived i.e., as any of SEQ ID NOs: 1-16. In some embodiments, fragments of a recombinant DNA molecule comprising a DNA sequence of any one of SEQ ID NOs: 1-16 are provided comprising at least about 50, at least about 75, at least about 95, at least about 100, at least about 125, at least about 150, at least about 175, at least about 200, at least about 225, at least about 250, at least about 275 contiguous nucleotides, or longer, of the reference sequence from which they are derived i.e., of any of SEQ ID NOs: 1-16, and having promoter activity as disclosed herein. In some embodiments, fragments of a promoter are provided comprising at least about 50, at least about 75, at least about 95, at least about 100, at least about 125, at least about 150, at least about 175, at least about 200, at least about 225, at least about 250, or at least about 275 contiguous nucleotides, of a DNA sequence comprising a TATA box and having at least about 85 percent identity, at least about 86 percent identity, at least about 87 percent identity, at least about 88 percent identity, at least about 89 percent identity, at least about 90 percent identity, at least about 91 percent identity, at least about 92 percent identity, at least about 93 percent identity, at least about 94 percent identity, at least about 95 percent identity, at least about 96 percent identity, at least about 97 percent identity, at least about 98 percent identity, at least about 99 percent identity, or at least about 100 percent identity to any of SEQ ID NOs: 1-16, and having promoter activity as disclosed herein. Methods for producing such fragments from a starting promoter molecule i.e., a reference sequence are well known in the art.

As used herein, an “expression cassette” refers to a polynucleotide sequence comprising at least a first polynucleotide sequence capable of initiating transcription of an operably linked second polynucleotide sequence and optionally a transcription termination sequence operably linked to said second polynucleotide sequence.

A palindromic sequence is a nucleic acid sequence that is the same whether read 5′ to 3′ on one strand or 3′ to 5′ on the complementary strand with which it forms a double helix. A nucleotide sequence is said to be a palindrome if it is equal to its reverse complement. A palindromic sequence can form a hairpin.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability.

Having described the principles of the present disclosure in detail, it will be apparent that modifications, variations, and equivalent embodiments are possible without departing from such principles. Furthermore, it should be appreciated that all examples in the present disclosure are provided as non-limiting examples.

### EMBODIMENTS

For further illustration, additional exemplary, non-limiting embodiments of the present disclosure are set forth below.

Embodiment 1 relates to a recombinant DNA molecule comprising a DNA sequence selected from the group consisting of: a) a sequence with at least 85% sequence identity to any of SEQ ID NOs: 1-16; b) a sequence comprising any of SEQ ID NOs: 1-16; and c) a fragment of any of SEQ ID NOs: 1-16, wherein the fragment comprises gene regulatory activity.

Embodiment 2 relates to the recombinant DNA molecule of embodiment 1, wherein said sequence has at least 90 percent sequence identity to the DNA sequence of any of SEQ ID NOs: 1-16.

Embodiment 3 relates to the recombinant DNA molecule of embodiments 1 or 2, wherein said sequence has at least 95 percent sequence identity to the DNA sequence of any of SEQ ID NOs: 1-16.

Embodiment 4 relates to the recombinant DNA molecule of any one of embodiments 1 to 3, wherein the DNA sequence comprises gene regulatory activity.

Embodiment 5 relates to the recombinant DNA molecule of any one of embodiments 1 to 4, wherein the DNA sequence comprises a synthetic small nuclear RNA (snRNA) promoter.

Embodiment 6 relates to the recombinant DNA molecule of any one of embodiments 1 to 5, wherein the DNA sequenced is operably linked to a heterologous transcribable DNA molecule.

Embodiment 7 relates to the recombinant DNA molecule of embodiment 6, wherein the heterologous transcribable DNA molecule encodes a non-coding RNA.

Embodiment 8 relates to the recombinant DNA molecule of embodiment 7, wherein the non-coding RNA is selected from the group consisting of: a guide RNA (gRNA), a single-guide RNA (sgRNA), a crRNA, a pre-crRNA, a tracrRNA, a PEgRNA, a tagRNA, a microRNA (miRNA), a miRNA precursor, a small interfering RNA (siRNA), a small RNA (22-26 nt in length) and precursor encoding same, a heterochromatic siRNA (hc-siRNA), a Piwi-interacting RNA (piRNA), a hairpin double-strand RNA (hairpin dsRNA), a trans-acting siRNA (ta-siRNA), and a naturally occurring antisense siRNA (nat-siRNA).

Embodiment 9 relates to the recombinant DNA molecule of embodiments 7 or 8, wherein the non-coding RNA is a gRNA.

Embodiment 10 relates to a recombinant DNA construct comprising at least a first expression cassette comprising a recombinant DNA molecule of any one of embodiments 1 to 5, operably linked to a DNA sequence encoding a gRNA.

Embodiment 11 relates to a recombinant DNA construct of embodiment 10, further comprising at least a second or more expression cassettes, wherein each of the expression cassettes comprises a recombinant DNA molecule of any one of embodiments 1 to 5, operably linked to a DNA sequence encoding a gRNA.

Embodiment 12 relates to the recombinant DNA construct of embodiment 11, wherein the DNA sequence encoding the gRNAs are identical or distinct from each other.

Embodiment 13 relates to the recombinant DNA construct of any one of embodiments 11 to 12, wherein the DNA sequences encoding the gRNAs target the same or different target sites in a chromosome of a plant cell.

Embodiment 14 relates to the recombinant DNA construct of any one of embodiments 11 to 13, wherein the recombinant DNA molecules of any one of embodiments 1 to 5 comprised therein, are identical or distinct from each other.

Embodiment 15 relates to the recombinant DNA construct of any one of embodiments 10 to 14, wherein the DNA molecules of any one of embodiments 1 to 5 comprised therein, comprise promoter activity.

Embodiment 16 relates to the recombinant DNA construct of any one of embodiments 10 to 15, wherein each expression cassette comprised therein, further comprises a transcription termination sequence.

Embodiment 17 relates to the recombinant DNA construct of any one of embodiments 10 to 16, further comprising flanking left and right homology arms (HA) each being about 200-1200 bp in length.

Embodiment 18 relates to the recombinant DNA construct of embodiment 17, wherein the homology arms are about 230 to 1003 bp in length.

Embodiment 19 relates to the recombinant DNA construct of any one of embodiments 10 to 18, further comprising a DNA sequence encoding a promoter operably linked to a DNA sequence encoding a Type I CRISPR-associated protein, a Type II CRISPR-associated protein, a Type III CRISPR-associated protein, a Type IV CRISPR-associated protein, Type V CRISPR-associated protein, or a Type VI CRISPR-associated protein.

Embodiment 20 relates to the recombinant DNA construct of embodiment 19, wherein the DNA sequence encoding the CRISPR-associated protein is further operably linked to at least one DNA sequence encoding a nuclear localization sequence (NLS).

Embodiment 21 relates to the recombinant DNA construct of any one of embodiments 19 to 20, wherein the CRISPR-associated protein is selected from the group consisting of: Cas1, Cas1B, Cas2, Cas3, Cas4, Cas5, Cas6, Cas7, Cas8, Cas9, Cas10, Cas12a, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, and Mad7, or wherein the CRISPR-associated protein is Cas12a.

Embodiment 22 relates to a transgenic cell comprising the recombinant DNA molecule of any one of embodiments 1 to 9 or the recombinant DNA construct of any one of embodiments 10 to 21.

Embodiment 23 relates to the transgenic cell of embodiment 22, wherein the cell is a transgenic plant cell.

Embodiment 24 relates to the transgenic plant cell of embodiment 23, wherein the plant cell is a monocotyledonous plant cell.

Embodiment 25 relates to the transgenic plant cell of embodiment 23, wherein the plant cell is a dicotyledonous plant cell.

Embodiment 26 relates to the transgenic plant cell of embodiment 23, wherein the plant cell is selected from the group consisting of: a maize plant cell, a soybean plant cell, a cotton plant cell, a peanut plant cell, a barley plant cell, an oat plant cell, an orchard grass plant cell, a rice plant cell, a sorghum plant cell, a sugarcane plant cell, a tall fescue plant cell, a turfgrass plant cell, a wheat plant cell, an alfalfa plant cell, a canola plant cell, a cabbage plant cell, a mustard plant cell, a rutabaga plant cell, a turnip plant cell, a kale plant cell, a broccoli plant cell, a cauliflower plant cell, a pepper plant cell, a bean plant cell, a cowpea plant cell, a chickpea plant cell, a gourd plant cell, a lettuce plant cell, a cucumber plant cell, a melon plant cell, a carrot plant cell, a tomato plant cell, a radish plant cell, a potato plant cell, and an ornamental plant cell.

Embodiment 27 relates to the recombinant DNA molecule of any one of embodiments 1 to 3, wherein said sequence has at least 99 percent sequence identity to the DNA sequence of any of SEQ ID NOs: 1-16.

The embodiments described herein may be more readily understood through reference to the following examples, which are provided by way of illustration, and are not intended to be limiting, unless specified. It should be appreciated by those of skill in the art that the techniques disclosed in the following examples represent techniques discovered by the inventors to function well in the practice of this disclosure. However, those of skill in the art should, in light of the disclosure, appreciate that many changes can be made in the specific embodiments that are disclosed and still obtain a like or similar result without departing from the principles of the disclosure, therefore all matter set forth or shown in the accompanying drawings is to be interpreted as illustrative and not in a limiting sense.

### EXAMPLES

The following examples are included to demonstrate embodiments of the disclosure. It should be appreciated by those of skill in the art that many changes can be made in the specific embodiments which are disclosed and still obtain a like or similar result without departing from the principles of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the scope and concept of the disclosure as defined by the appended claims.

### Example 1

### Synthesis of Promoters to Express gRNA

Novel synthetic transcriptional regulatory elements are synthetic expression elements designed through algorithmic methods. The synthetic promoter elements of the present disclosure provide transcription of small nuclear RNA (snRNA) molecules such as guide RNA (gRNA) molecules. The designed synthetic snRNA promoter elements do not have extended homology to any known nucleic acid sequences that exist in nature, yet affect transcription of an operably linked DNA sequence the same as naturally occurring snRNA promoters. The full-length synthetic snRNA promoters of the present disclosure share little sequence identity amongst each other; ranging from about thirty-eight (38) percent identity to about forty-five (45) percent identity. The low percentage identity amongst the synthetic snRNA promoters reduces the likelihood of recombination between promoters and makes the synthetic snRNA promoters ideal for stacking multiple RNA expression cassettes; in which each cassette comprises a different synthetic snRNA promoter. The synthetic snRNA promoters demonstrated the ability to drive expression of a gRNA as will be described further in the examples below. Table 1 below shows the different synthetic snRNA promoters and the respective length of each synthetic snRNA promoter.

TABLE 1
Synthetic snRNA promoters.

| Synthetic snRNA | SEQ ID | Length |
|---|---|---|
| Promoters | NO: | (bp) |
| P-GSP2241 | 1 | 300 |
| P-GSP2242 | 2 | 300 |
| P-GSP2243 | 3 | 300 |
| P-GSP2247 | 4 | 300 |
| P-GSP2248 | 5 | 300 |
| P-GSP2249 | 6 | 300 |
| P-GSP2250 | 7 | 300 |
| P-GSP2251 | 8 | 300 |
| P-GSP2253 | 9 | 300 |
| P-GSP2254 | 10 | 300 |
| P-GSP2255 | 11 | 300 |
| P-GSP2256 | 12 | 300 |
| P-GSP2258 | 13 | 300 |
| P-GSP2259 | 14 | 300 |
| P-GSP2260 | 15 | 300 |
| P-GSP2261 | 16 | 300 |

### Example 2

### Assay of the Synthetic snRNA Promoters in Driving Expression of a gRNA that Targets the Zm7 Genomic Locus Using Transfected Corn Protoplasts

Corn leaf protoplasts were transfected with constructs i.e., recombinant DNA constructs, a first construct comprising an expression cassette for the expression of Cas12a driven by a plant expressible promoter and a second construct comprising an expression cassette for the expression of gRNAs designed to target the Zm7 genomic locus driven by a synthetic snRNA promoter; and assessed for the effectiveness of inducing edits within the Zm7.1c target site sequence (SEQ ID NO: 23).

Corn leaf protoplasts were transfected with multiple constructs to assay the capacity of the synthetic snRNA promoters in driving expression of a gRNA, resulting in editing of a specific sequence within the Zm71.c target site sequence (SEQ ID NO:23), a single-copy intergenic site in the corn genome. Each protoplast preparation was transfected simultaneously with 4 different constructs. A first construct was used to drive the expression of Cas12a (Cas12a_NLS, SEQ ID NO: 20) in the protoplast cell using a constitutive promoter. The expression cassette within the first construct comprised the Zea mays Mexicana constitutive promoter, leader, and intron, EXP-Zm.UbqM1:1:9 (SEQ ID NO:19), operably linked 5′ to a coding sequence, Cas12a_NLS (SEQ ID NO: 20) encoding a nuclear targeted Cas12a_NLS protein (SEQ ID NO:22), operably linked 5′ to a 3′ UTR, T-Os.LTP: 2 (SEQ ID NO:21). A second construct was used to drive expression of a gRNA, gRNA_Zm7.1c (SEQ ID NO:17) which directs the Cas12a_NLS protein to cut within the Zm71.c target site sequence (SEQ ID NO:23), driven by a synthetic snRNA promoter selected from the group consisting of SEQ ID NOs: 1-16. A third and fourth construct were used to drive the expression of the Renilla and Firefly luciferase genes, respectively using constitutive promoters to assess the success of protoplast transfection.

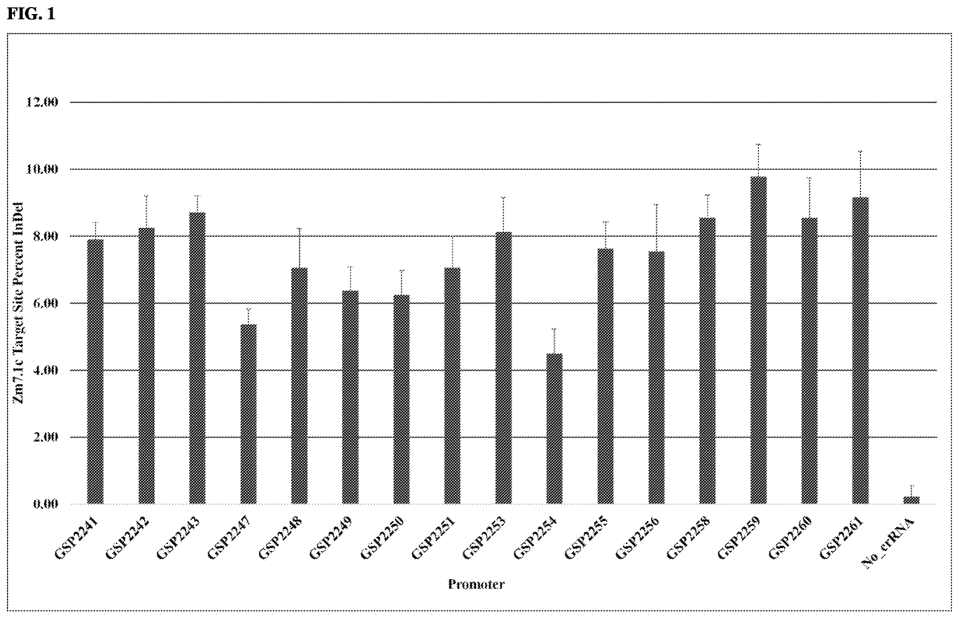

Corn leaf protoplasts were transfected using a PEG-based transfection method, similar to those known in the art, with the 4 types of constructs described above (first, second, third, and fourth). A control transfection used only 3 types of constructs (first, third, and fourth) and did not use the gRNA construct and is annotated as “No_crRNA”. Genomic DNA was isolated from the protoplast cells after transfection and incubation. DNA sequencing was performed around the Zm7.1c target site. Each transfection was repeated 4 times and an average % InDel (Insertion/Deletion) and standard deviation was calculated based upon the 4 replicates. Tables 2 and FIG. 1 show the mean percent InDel and standard deviation (St.Dev.) induced by gRNA_Zm7.1c driven by the synthetic snRNA promoters.

TABLE 2
Mean percent InDel and standard deviation induced by
gRNA_Zm7.1c driven by the synthetic snRNA promoters.

| Synthetic snRNA | SEQ ID | Percent | |
|---|---|---|---|
| Promoter | NO: | InDel | St. Dev. |
| GSP2241 | 1 | 7.9 | 0.50 |
| GSP2242 | 2 | 8.25 | 0.95 |
| GSP2243 | 3 | 8.71 | 0.49 |
| GSP2247 | 4 | 5.37 | 0.46 |
| GSP2248 | 5 | 7.06 | 1.17 |
| GSP2249 | 6 | 6.38 | 0.71 |
| GSP2250 | 7 | 6.25 | 0.73 |
| GSP2251 | 8 | 7.06 | 0.95 |
| GSP2253 | 9 | 8.13 | 1.03 |
| GSP2254 | 10 | 4.5 | 0.73 |
| GSP2255 | 11 | 7.63 | 0.80 |
| GSP2256 | 12 | 7.55 | 1.40 |
| GSP2258 | 13 | 8.56 | 0.67 |
| GSP2259 | 14 | 9.78 | 0.96 |
| GSP2260 | 15 | 8.55 | 1.19 |
| GSP2261 | 16 | 9.16 | 1.37 |
| No_crRNA | none | 0.23 | 0.33 |

As can be seen in Table 2 above and FIG. 1, all of the synthetic snRNA promoters were able to drive expression of gRNA_Zm7.1c to guide Cas12a_NLS to cut within the Zm7.1c target site resulting in insertions and deletions after repair of the cut site.

### Example 3

### Assay of the Synthetic snRNA Promoters in Driving Expression of a gRNA that Targets the Bmr3 Genomic Locus Using Transfected Corn Protoplasts

Corn leaf protoplasts were transfected with constructs, a first construct comprising an expression cassette for the expression of Cas12a driven by a plant expressible promoter and a second construct comprising an expression cassette for the expression of a gRNA designed to target the Bmr3 genomic locus driven by a synthetic snRNA promoter; and assessed for the effectiveness of inducing edits within the Bmr3_3170 target site sequence (SEQ ID NO:24).

Corn leaf protoplasts were transfected with multiple constructs to assay the capacity of the synthetic snRNA promoters in driving expression of a gRNA, resulting in editing of specific sequence within the brown midrib, Bmr3_3170 target site sequence (SEQ ID NO:24). The brown midrib mutations are among the earliest described in maize. Plants containing a brown midrib mutation exhibit a reddish-brown pigmentation of the leaf midrib starting when there are four to six leaves. These mutations are known to alter lignin composition and digestibility of plants and therefore constitute prime candidates in the breeding of silage maize. The Bmr3 gene encodes the enzyme O-methyltransferase (COMT) involved in lignin biosynthesis (Vignols et al., 1995, The Plant Cell, Vol. 7, 407-416).

Each protoplast preparation was transfected with 4 different constructs. A first construct was used to drive the expression of Cas12a (Cas12a_NLS, SEQ ID NO:20) in the protoplast cell using a constitutive promoter. The expression cassette within the first construct comprised the Zea mays Mexicana constitutive promoter, leader, and intron, EXP-Zm.UbqM1:1:9 (SEQ ID NO:19), operably linked 5′ to a coding sequence, Cas12a_NLS (SEQ ID NO:20) encoding a nuclear targeted Cas12a_NLS protein (SEQ ID NO:22), operably linked 5′ to a 3′ UTR, T-Os.LTP: 2 (SEQ ID NO: 21). A second construct was used to drive expression of a gRNA, gRNA_Bmr3_3170 (SEQ ID NO: 18) which directs the Cas12a_NLS protein to cut within the Bmr3_3170 target site sequence (SEQ ID NO:24), driven by a synthetic snRNA promoter selected from the group consisting of SEQ ID NOs: 1-16. A third and fourth construct were used to drive the expression of the Renilla and Firefly luciferase genes, respectively using constitutive promoters to assess the success of protoplast transfection.

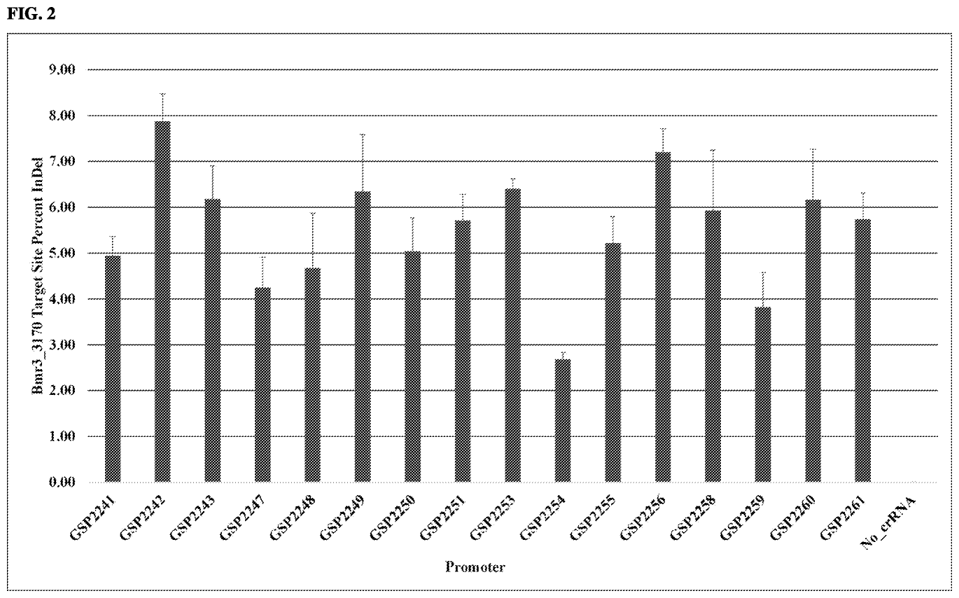

Corn leaf protoplasts were transfected using a PEG-based transfection method, similar to those known in the art, with the 4 types of constructs described above (first, second, third, and fourth). A control transfection used only 3 types of constructs (first, third, and fourth) and did not use the gRNA construct and is annotated as “No_crRNA”. Genomic DNA was isolated from the protoplast cells after transfection and incubation. DNA sequencing was performed around the Bmr3_3170 target site. Each transfection was repeated 4 times and an average % InDel (Insertion/Deletion) and standard deviation was calculated based upon the 4 replicates. Tables 3 and FIG. 2 show the mean percent InDel and standard deviation (St.Dev.) induced by gRNA_Bmr3_3170 driven by the synthetic snRNA promoters.

TABLE 3
Mean percent InDel and standard deviation induced by
gRNA_Bmr3_3170 driven by the synthetic snRNA promoters.

| Synthetic snRNA | SEQ | Percent | |
|---|---|---|---|
| Promoter | ID NO: | InDel | St. Dev. |
| GSP2241 | 1 | 4.94 | 0.42 |
| GSP2242 | 2 | 7.88 | 0.59 |
| GSP2243 | 3 | 6.18 | 0.72 |
| GSP2247 | 4 | 4.25 | 0.66 |
| GSP2248 | 5 | 4.68 | 1.19 |
| GSP2249 | 6 | 6.34 | 1.25 |
| GSP2250 | 7 | 5.04 | 0.73 |
| GSP2251 | 8 | 5.71 | 0.57 |
| GSP2253 | 9 | 6.40 | 0.22 |
| GSP2254 | 10 | 2.69 | 0.15 |
| GSP2255 | 11 | 5.22 | 0.58 |
| GSP2256 | 12 | 7.20 | 0.52 |
| GSP2258 | 13 | 5.93 | 1.31 |
| GSP2259 | 14 | 3.83 | 0.75 |
| GSP2260 | 15 | 6.16 | 1.10 |
| GSP2261 | 16 | 5.74 | 0.57 |
| No crRNA | none | 0.00 | 0.00 |

As can be seen in Table 3 above and FIG. 2, all of the synthetic snRNA promoters were able to drive expression of gRNA_Bmr3 to guide Cas12a_NLS to cut within the Bmr3_3170 target site resulting in insertions and deletions after repair of the cut site.

Having illustrated and described the principles of the presentdisclosure, it should be apparent to persons skilled in the art that the disclosure can be modified in arrangement and detail without departing from such principles. All publications and published patent documents cited herein are hereby incorporated by reference to the same extent as if each individual publication or patent application is specifically and individually indicated to be incorporated by reference.

1. A recombinant DNA molecule comprising a DNA sequence selected from the group consisting of:
a. a sequence with at least 85% sequence identity to any of SEQ ID NOs: 1-16;
b. a sequence comprising any of SEQ ID NOs: 1-16; and
c. a fragment of any of SEQ ID NOs: 1-16, wherein the fragment comprises gene regulatory activity.

2. The recombinant DNA molecule of claim 1, wherein said sequence has at least 90 percent sequence identity to the DNA sequence of any of SEQ ID NOs: 1-16.

3. The recombinant DNA molecule of claim 1, wherein said sequence has at least 95 percent sequence identity to the DNA sequence of any of SEQ ID NOs: 1-16.

4. The recombinant DNA molecule of claim 1, wherein the DNA sequence comprises gene regulatory activity.

5. The recombinant DNA molecule of claim 1, wherein the DNA sequence comprises a synthetic small nuclear RNA (snRNA) promoter.

6. The recombinant DNA molecule of claim 1, wherein the DNA sequence is operably linked to a heterologous transcribable DNA molecule.

7. The recombinant DNA molecule of claim 6, wherein the heterologous transcribable DNA molecule encodes a non-coding RNA.

8. The recombinant DNA molecule of claim 7, wherein the non-coding RNA is selected from the group consisting of: a guide RNA (gRNA), a single-guide RNA (sgRNA), a crRNA, a pre-crRNA, a tracrRNA, a PEgRNA, a microRNA (miRNA), a miRNA precursor, a small interfering RNA (siRNA), a small RNA (22-26 nt in length) and precursor encoding same, a heterochromatic siRNA (hc-siRNA), a Piwi-interacting RNA (piRNA), a hairpin double-strand RNA (hairpin dsRNA), a trans-acting siRNA (ta-siRNA), and a naturally occurring antisense siRNA (nat-siRNA); or wherein the non-coding RNA is a gRNA.

9. A recombinant DNA construct comprising at least a first expression cassette comprising a first recombinant DNA molecule of claim 1, operably linked to a DNA sequence encoding a gRNA.

10. The recombinant DNA construct of claim 9, further comprising at least a second or more expression cassettes, wherein each of the expression cassettes comprise a recombinant DNA molecule of claim 1, operably linked to a DNA sequence encoding a gRNA.

11. The recombinant DNA construct of claim 10, wherein the DNA sequences encoding the gRNAs are identical or distinct from each other.

12. The recombinant DNA construct of claim 10, wherein the DNA sequences encoding the gRNAs target the same or different target sites in a chromosome of a plant cell.

13. The recombinant DNA construct of claim 10, wherein the recombinant DNA molecules of claim 1 comprised in the at least two expression cassettes comprised therein, are identical or distinct from each other.

14. The recombinant DNA construct of claim 9, wherein the recombinant DNA molecule of claim 1 comprised in the at least first expression cassette, comprises promoter activity and/or, wherein the at least first expression cassette comprised in the recombinant DNA construct further comprises a transcription termination sequence.

15. The recombinant DNA construct of claim 9, further comprising a DNA sequence encoding a promoter operably linked to a DNA sequence encoding a Type I CRISPR-associated protein, a Type II CRISPR-associated protein, a Type III CRISPR-associated protein, a Type IV CRISPR-associated protein, Type V CRISPR-associated protein, or a Type VI CRISPR-associated protein.

16. The recombinant DNA construct of claim 15, wherein the DNA sequence encoding the CRISPR-associated protein is further operably linked to at least one DNA sequence encoding a nuclear localization sequence (NLS).

17. The recombinant DNA construct of claim 15, wherein the CRISPR-associated protein is selected from the group consisting of: Cas1, Cas1B, Cas2, Cas3, Cas4, Cas5, Cas6, Cas7, Cas8, Cas9, Cas10, Cas12a, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, and Mad7, or wherein the CRISPR-associated protein is Cas12a.

18. A transgenic plant cell comprising the recombinant DNA molecule of claim 1 or the recombinant DNA construct of claim 9.

19. The transgenic plant cell of claim 18, wherein the plant cell is a monocotyledonous plant cell.

20. The transgenic plant cell of claim 18, wherein the plant cell is a dicotyledonous plant cell.


SEQUENCE LISTING (24 sequences)

SEQ ID NO: 1 - DNA, 300 bp, synthetic construct
  source 1..300: mol_type = other DNA; note = DNA sequence of the synthetic snRNA promoter, P-GSP2241.
ctacgtacttctttttcttctacttgagcaagtgtgtgcaccgcgttttttagcgttcgcacgttcacagcccagctgggcccaatttgtttagtgtgtatcctgagccaaaaagcccaatcagcccactgggccgagtgctgggcctccgcgcgggaagtaccgcgctgcgagaaaagggcggtttgggtgtaaggcgcttaacgcgcggaggaaggtttaagcgggggcgtttagtcccatctcggccagccagcaacgcgcagaccagcttataagcttaccgagctgctttccgtc

SEQ ID NO: 2 - DNA, 300 bp, synthetic construct
  source 1..300: mol_type = other DNA; note = DNA sequence of the synthetic snRNA promoter, P-GSP2242.
gttttcttatctacggttttatacaaaaagaggtataataacagtgcaatcgcgagatagtactttagtaatagagaaagcactcggaggagcccaacaagggcaaaaagccaaaagcccagcccaatactgggcctgggcccacagacgcgcgggcgcgggccgtgcgctgcggccccgaggctggttcctgcggagcgttgctgggagcaagcggggcaacaggaatcgtttagtaccacatcggcaacgcaaacgaccagcgaccagtttataacgctgccagagcggagcgctgtc

SEQ ID NO: 3 - DNA, 300 bp, synthetic construct
  source 1..300: mol_type = other DNA; note = DNA sequence of the synthetic snRNA promoter, P-GSP2243.
aggttaattgaaaaagaagtgtgtgacttacaacgattacacagtacagaatacagtgtcaggccccaacggtgggagaaaaggcgtaggcctgggttagtggactgctgggcctctctgggtacggagaaagccgaagaaacaagaagaaggccgtgtaccgcgcggcgctggcgcagaaggcccaattcgcgctctagcgcggggcaacgagcggggcaacgcgctaagtttagtaccacatcggaaaccgaacagctaacgacccaccttataaggtggtaactgcagctgccactc

SEQ ID NO: 4 - DNA, 300 bp, synthetic construct
  source 1..300: mol_type = other DNA; note = DNA sequence of the synthetic snRNA promoter, P-GSP2247.
cagagtttttatacctgtgtgtaaaatacaacttctgtgtacttgagatagcaccgtccccggcccagcaaaaattctggcaaaaaaagaggcttcccactaattgagaagaaaagaagaggcctaagaaacgcccgtgggcccacaaggcgggccgctcgagcgcggcccactttcgagcagttcgtcgaacgagagcgcggggagcgcgggaacagtggagaggactcagtttagtcccatctcgagcaaggaaacagaccaagaccactttataacgtagcctgctctccacccttc

SEQ ID NO: 5 - DNA, 300 bp, synthetic construct
  source 1..300: mol_type = other DNA; note = DNA sequence of the synthetic snRNA promoter, P-GSP2248.
aattcttatttcttttagtaccttcttctgggggcggcgttcgcagaaagaatacgttcgcacgtagcgaaaagggccgaaaaaggaaagcctggaccaaggaaaaacacaaaaagcgtatcctggaaaggccgcttcggtttagcccgtgtgcgggggagcagcccaatacactttagcccaaattagcgcgggtcggtggaacgcggggcaacaggcggaagtttagtcgtttagtaccatatcggaaattaagagcgaacagacccactttataacgctgtagctaccaccctggtc

SEQ ID NO: 6 - DNA, 300 bp, synthetic construct
  source 1..300: mol_type = other DNA; note = DNA sequence of the synthetic snRNA promoter, P-GSP2249.
tagtacggctgaatcgtgtctccagcagaccgggcacagtgtgtcaagcgttgacgtcaagcgttcgccgcaggcctggcccaatctgggtcaacagggcaaagccctcgcaagaaaagaaagaaagaagccagcccaatcggcccagcccacgcctcaacaacgagcccactgcggccttgcgggagcgtccttcggtcggtggagcgcgttgcggggtttagtacctcagtttagtaccatatcggcttagcaaggaacaaggaaccagtttataagctgcagctgtgctgcctggtc

SEQ ID NO: 7 - DNA, 300 bp, synthetic construct
  source 1..300: mol_type = other DNA; note = DNA sequence of the synthetic snRNA promoter, P-GSP2250.
cgtgtgaccggcgcacttaaaacacacgggcgtgtgtcaccagaggtatatcaagcgcgcaatacgcacgtaggtccgctgtatttgttaaaaaatcagctacttgttgttgtttgctccgttgggccgcagcgaagaagatttggcgggcttcagcccagcccgcagcttgcgggcgcttccgcgagcggcccacttcggtggagcgcaacgcgcggggtttaagcgttgtttagtcccatctcggaggcaaagctagcgaaagaccaccttataaccgagcggaagcgctgcttgatc

SEQ ID NO: 8 - DNA, 300 bp, synthetic construct
  source 1..300: mol_type = other DNA; note = DNA sequence of the synthetic snRNA promoter, P-GSP2251.
ccagcaccagcagcaccagagttcgtgaaaccttcttctgttcgtcaccagaactcttcttcttctgtgtgtgtgcaaaatctgggttacaacaagggcgtgtggcgtatcctcacaggaaagaaagcgaaccttgggccccgagcgggccggaagtaccgccacaaagcccacgaggcccacttcggttcgtctcgtcgcgcttacgcgcgcggggtttaacaacagaggtttagtaccatatcggaaacacaaagcgaagcgtaccaccttataaggaaagcgaagcgaagcgcgctc

SEQ ID NO: 9 - DNA, 300 bp, synthetic construct
  source 1..300: mol_type = other DNA; note = DNA sequence of the synthetic snRNA promoter, P-GSP2253.
atttttaaccggttctttaacagagtgaagaagtgtcagaaagctcagtaggagccgcgagaatagtcagaggagaaccgagaagcctagtgttaaaggcttgctgggctgtgcaaagaagaaagcccacttgggcctgggcctcaacaagcccaatacgaaaggctgggcgcgctgcggggggcttgcgggcttcgtcggcgcgcttgggggcaacgagcagaggtttagtttagtaccatatcgcctcgaaaggcaaggaacgaccaccttataacgctgcagctgtgcagcttaatc

SEQ ID NO: 10 - DNA, 300 bp, synthetic construct
  source 1..300: mol_type = other DNA; note = DNA sequence of the synthetic snRNA promoter, P-GSP2254.
gtttttttcttctttttttcttttttctacccttctacgccaggagataagtgtgttcgcacgcttcacctgtacaaaaaagcaactgggctccagcaacgagagccacaatcaacttcgttaagaagcaagaaagatctccgctagcccgtgtactcgcttccgtcgggaggcccactctggcgcgctgcggggagcggcttgcaacgcggggcgggggcgctagaacaggtttagtaccatatcgagcagttaacgcagcaacgaccactttataacccgccaaaccaccctgctctc

SEQ ID NO: 11 - DNA, 300 bp, synthetic construct
  source 1..300: mol_type = other DNA; note = DNA sequence of the synthetic snRNA promoter, P-GSP2255.
tttatatactgttcgctgtcgcttacagagccagaagtgtgtcagcgccgcgaggtgctggtgtacaagaagagctttgttaaaaaaaggcgtatcagcccactgggcccaccttgggctaaagcttataagcggcccaaagttgggcctccgcgggctctaccgaagcccacacgggcgggagcggcccagttcgcgctgggattgcgctgagcagagcgttaagcgttgtttagtaccatatcggctgctcacgcgaaagcgacccaccttataaggcgcttaaccggaagcctggtc

SEQ ID NO: 12 - DNA, 300 bp, synthetic construct
  source 1..300: mol_type = other DNA; note = DNA sequence of the synthetic snRNA promoter, P-GSP2256.
caaaggcacaccctcgtgaaacaaagcaacaaagaggcaacagtttttagctggattacagcaagaagcgcgcttcagctgtatctggcggcctggactgagcaatcagctgctgatttgcaaaagcggccgtgcacaagaaacaggccgtgggctgctacaactgggctggcccaccgcgggctggccagttcggtagcgcttacgagcaagcgggggcaacagagtttgtttagtaccacatcggttgctcagcgcaaacgcgaccagcttataacgctttggcaccgttgcgggatc

SEQ ID NO: 13 - DNA, 300 bp, synthetic construct
  source 1..300: mol_type = other DNA; note = DNA sequence of the synthetic snRNA promoter, P-GSP2258.
ttgcgcttcgctgccaaaattgctgtcaacggttcggagtgcagatagtcctgtttttttgtagccgcgttcggagaactgcaaaaaaatcagctgacgaggctctgatcgaggaagaaaggccttgggccgtttcttcggtttagaagcccacgcgggctgtgggcgctgggctgcggcgctgcgctgcagtagtacccagtaacgcagtaacgcgcaaacgtttagtagtttagtaccatctcggctgccgaaagctaacgaagccactttataagcctcgcgcggaagcaccagttc

SEQ ID NO: 14 - DNA, 300 bp, synthetic construct
  source 1..300: mol_type = other DNA; note = DNA sequence of the synthetic snRNA promoter, P-GSP2259.
aaaggagcaaaggaatcgttttcagagtttacaaattgagcgaatcagcccaagaatacgtgtcaaagacttgagcttatctggtattataaacctggtaaaggggggagaaagccacttgggcccagcctagaaagcaaggtgaggaaaggcgcggctggagagtgctggcccaatacagttcgttccgggctggggagcgcaagcgcttacgcggggcaacgagcagagtttagtaccacctcggttgctcaacgcaaaggaacccaccttataaccacttataggcgctagctgctc

SEQ ID NO: 15 - DNA, 300 bp, synthetic construct
  source 1..300: mol_type = other DNA; note = DNA sequence of the synthetic snRNA promoter, P-GSP2260.
ggtaagtttttcttttttctttttgccgtacagtgtgtgtcaagacctgtagcaacaaaattgagcttaaaagagtttactgggctctgaacagctctgagaaagcccacggtgggctgaggcctcctcaactgggcctagaaggcccagaaggcctgggctgggcttccagtacgcgggtttgggccaggcgggaggcaacgcaacgcagccagtgcgctaaaacgtttagtttagtaccacctcggctgcgtagaaagctaacgaccagcttataacgtagccgagcctcacagcgtc

SEQ ID NO: 16 - DNA, 300 bp, synthetic construct
  source 1..300: mol_type = other DNA; note = DNA sequence of the synthetic snRNA promoter, P-GSP2261.
tgcacttatttagtaccagcaaccaccagaggcggcaacagaaaacgttcttcaattgttgtgggctgtgttagaggagctgtgtgctctgaggctctgggctctaacaaaagctaaagaagagctgggccggcccaaagaaacaggccgtacacgcgctcggcccacgcgcttgctactggagcagtaaggcctgggctggcgcttgccggtggagagcagaggttaacagtttagtaccatctcggctgctcaagctaacgcagaccactttataagctggagctgccttgcagcgtc

SEQ ID NO: 17 - DNA, 51 bp, synthetic construct
  source 1..51: mol_type = other DNA; note = DNA sequence of a guide RNA, gRNA_Zm7.1c.
gaatttctactaagtgtagatgtataatatgatggcatgccctcttttttt

SEQ ID NO: 18 - DNA, 51 bp, synthetic construct
  source 1..51: mol_type = other DNA; note = DNA sequence of a guide RNA, gRNA_Bmr3_3170.
gaatttctactaagtgtagatgaatccacgacatgcaagagctcttttttt

SEQ ID NO: 19 - DNA, 2008 bp, synthetic construct
  source 1..2008: mol_type = other DNA; note = DNA sequence of an expression element group (EXP), EXP-Zm.UbqM1:1:9 comprised of a promoter, leader, and intron derived from a Zea mays ssp. mexicana ubiquitin gene.
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttctgcaggtc

SEQ ID NO: 20 - DNA, 3750 bp, synthetic construct
  source 1..3750: mol_type = other DNA; note = DNA sequence encoding a nuclear targeted Cas12a protein, Cas12a_NLS.
atggcgggatctaagaagagaagaattaaacaagattcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcacggatctaagaagagaagaattaaacaagattga

SEQ ID NO: 21 - DNA, 298 bp, Oryza sativa
  source 1..298: mol_type = genomic DNA; note = DNA sequence of the 3' UTR, T-Os.LTP:2.
taatcgatcctccgatcccttaattaccataccattacaccatgcatcaatatccatatatatataaaccctttcgcacgtacttatactatgttttgtcatacatatatatgtgtcgaacgatcgatctatcactgatatgatatgattgatccatcagcctgatctctgtatcttgttatttgtataccgtcaaataaaagtttcttccacttgtgttaataattagctactctcatctcatgaaccctatatataactagtttaatttgctgtcaattgaacatgatgatcgatg

SEQ ID NO: 22 - protein, 1249 aa, synthetic construct
  source 1..1249: note = Amino acid sequence of a nuclear targeted Cas12a protein encoded by SEQ ID NO:20.
MAGSKKRRIKQDSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHGSKKRRIKQD

SEQ ID NO: 23 - DNA, 476 bp, Zea mays
  source 1..476: mol_type = genomic DNA; note = DNA sequence of the Zea mays Zm7 genomic region targeted for genome editing.
gtcaagagcaagcaacgaatatgctgtgctactcctctacgtgcggagacgtgttgtattttattctatgagcgttatttactgacgtaaggtatggtttaatcgacggattgctatcctggtcgggcttaattaaggacgttgaagcctggtaaggtatactaacttagcgtccttcggaatgggagtgagttgggaggacttgaaaatgtaagaagaagaaaacaaagcatgtgatggatgcttctggatgtgcaagctcagtcttcattcttcagcagcgcggaaattggctctaataacattggtttatgttgacagtgctagcgaggacaatgagtcattcgatgtgaaataataccatctgttctttatgatagtctatttagtataatatgatggcatgccctcttaggcagtagccggccagcatttgaaacatggtacttcgttttatttaaaaaataataagttat

SEQ ID NO: 24 - DNA, 3955 bp, Zea mays
  source 1..3955: mol_type = genomic DNA; note = DNA sequence of the Zea mays brown midrib 3 (Bmr3) genomic region targeted for genome editing.
ataaaaagttgtagatcttaaaaagttattaaactttatagttagttagcaactttttcgtttaaattcgtttagtgctacaaacaatcgatttatattcggtttgttttaatatgcggacaacaaacctataatctaaacataagttaaagttagtgatgggtaaagtggtagagaagtgaggttagagtttcgattcgcgaaaggtcctacaacaaacctaaaatctttttttgctatttattttaaaacatggtttatatttccaaaaaaatgtattggcggtttttaactcaggcgccaacaaaaattgatttccacggcagctgccaccgtcgttatcgctgaccaacccggctggtcgcctgtctgctccatccatgcatgttacaactatgcagatgcagccgaaacaaacactggctagaaaggcagccaacgggcctactgtcattcgctccggcatcctactggtgggcccagttgcaccggccgatgaccagttcatcatttttctcgacgaatttgtgcacagatcgaatttactaaaaattcttcgcacgtggcaaaaccaggggaaaaatcgagaactagtcgggtttttttatattccctgatagaatagtccctgctattcatccatgaaaaccaaacacgtacactacgtcaccgtcatggatgggagcgagtgaactgatgattttttccccaccccgcacgcaacagcatgggtgacaacaaccactcccgctgcggttgggcgagcacatctctacgcacttgacactcacgcaaacctaacgcatactagagtaatcatcgccaccaactatcggcgacagaaacgatgggccccgcttctcttaatcacggtgcttgaattagtgcgcgcatagtagtgaaaataatagtgaaaaataagcagtgcgtgttttggtgtggtggttggtgagccgtccggcccaataaaaaccctcgcaccacctcgtccctcttcgtcgcatcgcacgccatcagctagcagctagcgcgctcctcgagcccagcagagaaaggccggcagcgctaatcgtaatagccatgggctccaccgccggcgacgtggccgcggtggtggacgaggaggcgtgcatgtacgcgatgcagctggcgtcgtcgtccatcctgcccatgacgctgaagaacgccatcgagctgggcctgctggaggtgctgcagaaggaggccggcggcggcaaggcggcgctggcgcccgaggaggtggtggcgcggatgcccgcggcgcccggcgaccccgccgccgcggcggccatggtggaccgcatgctccgcctgctcgcctcctacgacgtcgtccggtgccagatggaggaccgggacggccggtacgagcgccgctactccgccgcgcccgtctgcaagtggctcacccccaacgaggacggcgtgtccatggccgccctcgcgctcatgaaccaggacaaggtcctcatggagagctggtgagtagtagccgcatcgcatcaaccaccttctacctatctatatccatcacttgttgctgctggcgtgcgcggcatgcatgatgacgagctcgctcatcattggtgctactagtgatttatttcgtccagtaaaattaattaaggtgcgctgctactctactggctgcggctagcacaaggctggaaatagttgttacttgttatacacgatataatatttctctagaacaaaaagatttttttttttataaaaagcaagcaagaaagaaagtgagtgacttcatgtttttcctaaaaaaaagttaggagtgggatggaaaagtcagcaaggaccacttgtttgttgtccactatccatccagtgggtgagacttttttgcgagacggagcactatattattggccgagtcctttttctgtatccgcaaaacggcagccgtcgatcgccggacggatcgacggctcacatgagtgtcgagtccaattccaaccacgagggcggcaaggaaaaccatccgtgctggtctggactttttgccaaactccattcagccattcgccgactgaaggtgaatcttcagacagccagattgtttggtgtctagtgtgtgcgaagatggcgtagaaaagactgagagacagttggctcacacagacaagtgacaactgactatagtatctgcctgcctggctgatgctgatagagatggggactcttgtcctgtctgtttcttgtatgcgctgatctgattctgatcactgccactctgccaggtactatctcaaggacgcggtgctggacggcggcatcccgttcaacaaggcgtacgggatgacggcgttcgagtaccacgggacggacccgcgcttcaaccgcgtgttcaacgagggcatgaagaaccactcggtgatcatcaccaagaagctgctggacttctacacgggcttcgagggcgtgtcgacgctggtggacgtgggcggcggcgtgggcgccacgctgcacgccatcacgtcccgccacccgcacatctccggggtcaacttcgacctgccgcacgtcatctccgaggcgccgccgttccccggcgtgcgccacgtgggcggggacatgttcgcgtccgtgcccgccggcgacgccatcctcatgaagtggatcctccacgactggagcgacgcgcactgcgccacgctgctcaagaactgctacgacgcgctgccggaaaatggcaaggtcatcgtcgtcgagtgcgtgctgccggtcaacacggaggccacccccaaggcgcagggcgtgttccacgtcgacatgatcatgctcgcgcacaacccaggcggcaaggagcggtacgagcgcgagttccgcgagctcgccaagggcgccggcttctccgggttcaaggccacctacatctacgccaacgcctgggccatcgagttcatcaagtgaatacggctaccaccgtcgccgcgatgagatgcatggctgccacatgcatgcttgcttgcttggtcctcgtatcgtacgtcgccgtcgtcgtcttcttctggttgcgctgctaccttgctgctctcgccctcgcgtatgcatgtacttttgcttaattttctttcttcatatcatgcactctggctggcctagactgcccccgatctatgctggccggtacgtcttgtcgagctcttgcatgtcgtggattctaaattcttcttctgcgtcgaattgtctctgccatgtgcgagtaataacaatcaaggttatacttaccatacaattacatggcggtttaattgctctcttttaatttggtgaagaatcgaatcgattctactgaattcttctctgtcgtttaatgaaatttcctttgtcttttggattatataatataacacagacactcacaaagacactcacaacgtgcgcacactcattcctataaacgcacacgcgcaaatcctactcctatcaagtatcaacactttcaagacttgaactggtcataccggagaaaatggactttcgttcgaggacataattacgtgttgctttagaaccaataccaactcaatcatcaacatcactttttaactgttagtatcggtttgtgtcttcaataagtactaatgttaagtcattaacaccggttgaatacaccaactgccgaagattgttcgacagtgaatatacgaggtatgtctatgaatgaaaacggacggaaacggtcggaaaaatcaatctcccatttcggtttccattttttatcaaaaacaggagcgggagcgatatagcaagaaatagaaaaggtagtgggatatacggttacacgaaaacggattaaaacaaacggatatgcaacgaaaatggatgctaatcggaaactttggctggaatatgcaatattttataatcactcaacgactacaatattaagagtttagaatactataaatca